(19) United States
(12) Patent Application Publication
(10) Pub. No.: US 2003/0096854 A1
(43) Pub. Date: May 22, 2003

(54) Substituted tricyclics

(76) Inventors: Ho-Shen Lin, Indianapolis, IN (US); Michael Enrico Richett, Indianapolis, IN (US)

Correspondence Address: ELI LILLY AND COMPANY, PATENT DIVISION, P.O. BOX 6288, INDIANAPOLIS, IN, 46206-6288, US

(21) Appl. No.: 10/168,152

(22) PCT Filed: Jan. 5, 2001
(86) PCT No.: PCT/US01/10850

(51) Int. Cl. A61K 31/403; C07D 209/82

(52) U.S. Cl. 514/411; 548/441

(57) ABSTRACT

A class of novel indole compounds is disclosed together with the use of such compounds for inhibiting sPLA₂ mediated release of fatty acids for treatment of Inflammatory Diseases such as septic shock

### FIELD OF THE INVENTION

This invention relates to novel substituted tricyclic organic compounds useful for inhibiting sPLA₂ mediated release of fatty acids for the treatment of inflammatory diseases.

### BACKGROUND INFORMATION

The structure and physical properties of human non-pancreatic secretory phospholipase A₂ (hereinafter called, “sPLA₂”) has been thoroughly described in two articles, namely, “Cloning and Recombinant Expression of Phospholipase A₂ Present in Rheumatoid Arthritic Synovial Fluid” by Seilhamer, Jeffrey J.; Pruzanski, Waldemar; Vadas Peter; Plant, Shelley; Miller, Judy A.; Kloss, Jean; and Johnson, Lorin K.; The Journal of Biological Chemistry, Vol. 264, No. 10, Issue of April 5, pp. 5335-5338, 1989; and “Structure and Properties of a Human Non-pancreatic Phospholipase A₂” by Kramer, Ruth M.; Hession, Catherine; Johansen, Berit; Hayes, Gretchen; McGray, Paula; Chow, E. Pingchang; Tizard, Richard; and Pepinsky, R. Blake; The Journal of Biological Chemistry, Vol. 264, No. 10, pp. 5768-5775, 1989; the disclosures of which are incorporated herein by reference.

It is believed that sPLA₂ is a rate limiting enzyme in the arachidonic acid cascade which hydrolyzes membrane phospholipids. Thus, it is important to develop compounds that inhibit sPLA₂ mediated release of fatty acids (e.g., arachidonic acid). Such compounds would be of value in the general treatment of conditions induced and/or maintained by overproduction of sPLA₂ such as septic shock, adult respiratory distress syndrome, pancreatitis, trauma-induced shock, bronchial asthma, allergic rhinitis, rheumatoid arthritis, etc.

It is desirable to develop new compounds and treatments for sPLA₂ induced diseases.

Alexander, et al., U.S. Pat. Nos. 3,939,177 and 3,979,391, disclose 1,2,3,4-tetrahydrocarbazoles useful as antibacterial agents. U.S. Pat. No 5,654,326 discloses compounds useful as anti-inflammatory agents. The enormity of the health problem associated with inflammation attributable to sPLA₂ mediated release of fatty acids necessitate a continuing search for alternate therapeutic agents. The present invention provides potent compounds useful as inhibitors of sPLA₂ mediated release of fatty acids.

### SUMMARY OF THE INVENTION

This invention provides tricyclic compounds as depicted in the general formula (I) below:

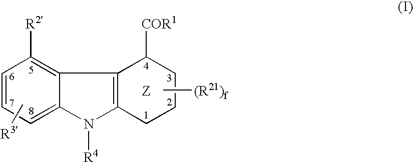

wherein;

Z is cyclohexenyl, or phenyl,

R⁴ is selected from groups (a), (b) and (c) where;

(a) is —(C₅-C₂₀)alkyl, —(C₅-C₂₀)alkenyl, —(C₅-C₂₀)alkynyl, carbocyclic radicals, or heterocyclic radicals, or

(b) is a member of (a) substituted with one or more independently selected non-interfering substituents; or

(c) is the group —(L)—R⁸⁰; where, (L)-is a divalent linking group of 1 to 12 atoms selected from carbon, hydrogen, oxygen, nitrogen, and sulfur; wherein the combination of atoms in —(L)— are selected from the group consisting of (i) carbon and hydrogen only, (ii) one sulfur only, (iii) one oxygen only, (iv) one or two nitrogen and hydrogen only, (v) carbon, hydrogen, and one sulfur only, and (vi) an carbon, hydrogen, and oxygen only; and where R⁸⁰ is a group selected from (a) or (b);

R²¹ is a non-interfering substituent where f is 1-3;

R¹ is —NHNH₂, —NH₂, or —CONH₂;

R^{2′} is selected from the group consisting of —O(CH₂)ₜR^{5′}

where

R^{5′} is —CONR⁹R¹⁰, where R⁹ and R¹⁰ is independently H, —(C₁-C₆)alkyl, —(C₁-C₆)alkyl substituted with —CO₂H or a derivative thereof, or —CF₃; phenyl or phenyl substituted with —(C₁-C₆)alkyl; t is 1-5; or R^{5′} is —(Lₕ)-(acylamino acid) wherein —(Lₕ)— is an acylamino acid linker having an acylamino-acid linker length of 1 to 7;

R^{3′} is selected from non-interfering substituent, carbocyclic radicals, carbocyclic radicals substituted with non-interfering substituents, heterocyclic radicals, and heterocyclic radicals substituted with non-interfering substituents;

or a pharmaceutically acceptable racemate, solvate, tautomer, optical isomer, prodrug derivative or salt, thereof.

The substituted tricyclic compounds of the present invention are effective in inhibiting human sPLA₂ mediated release of fatty acids.

This invention is also a pharmaceutical formulation comprising a compound of formula I in association with one or more pharmaceutically acceptable diluents, carriers and excipients.

This invention is also a method of inhibiting sPLA₂ comprising administering to a mammal in need of such treatment a therapeutically effective amount of a compound of formula I.

According to a further aspect of the present invention, there is provided a method of selectively inhibiting sPLA₂ in a mammal in need of such treatment comprising administering to said mammal a therapeutically effective amount of a compound of formula I.

This invention, further provides a compound of formula I for use as a medicament in the treatment of inflammatory diseases such as septic shock, adult respiratory distress syndrome, pancreatitis, trauma-induced shock, bronchial asthma, allergic rhinitis, rheumatoid arthritis, cystic fibrosis, stroke, acute bronchitis, chronic bronchitis, acute bronchiolitis, chronic bronchiolitis, osteoarthritis, gout, spondylarthropathris, ankylosing spondylitis, Reiter's syndrome, psoriatic arthropathy, enterapathric spondylitis, Juvenile arthropathy or juvenile ankylosing spondylitis, Reactive arthropathy, infectious or post-infectious arthritis, gonoccocal arthritis, tuberculous arthritis, viral arthritis, fungal arthritis, syphilitic arthritis, Lyme disease, arthritis associated with “vasculitic syndromes”, polyarteritis nodosa, hypersensitivity vasculitis, Luegenec's granulomatosis, polymyalgin rheumatica, joint cell arteritis, calcium crystal deposition arthropathris, pseudo gout, non-articular rheumatism, bursitis, tenosynomitis, epicondylitis (tennis elbow), carpal tunnel syndrome, repetitive use injury (typing), miscellaneous forms of arthritis, neuropathic joint disease (charco and joint), hemarthrosis (hemarthrosic), Henoch-Schonlein Purpura, hypertrophic osteoarthropathy, multicentric reticulohistiocytosis, arthritis associated with certain diseases, surcoilosis, hemochromatosis, sickle cell disease and other hemoglobinopathries, hyperlipoproteineimia, hypogammaglobulinemia, hyperparathyroidism, acromegaly, familial Mediterranean fever, Behat's Disease, systemic lupus erythrematosis, or relapsing polychondritis and related diseases which comprises administering to a mammal in need of such treatment a therapeutically effective amount of the compound of formula I in an amount sufficient to inhibit sPLA₂ mediated release of fatty acid and to thereby inhibit or prevent the arachidonic acid cascade and its deleterious products.

This invention provides, in addition, a process for preparing compounds of formula II

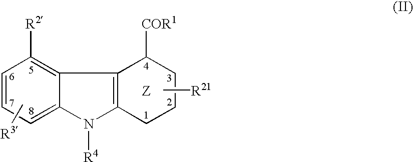

wherein;

Z is cyclohexenyl, or phenyl,

R²¹ is a non-interfering substituent;

R¹ is —CONH₂, —NHNH₂ or —NH₂;

R^{2′} is the group —O(CH₂)ₜR^{5′} where

R^{5′} is —CONR⁹R¹⁰, where R⁹ and R¹⁰ are independently H, —(C₁-C₆)alkyl or —CF₃; phenyl or phenyl substituted with —(C₁-C₆)alkyl; t is 1-5; or R^{5′} is the group —(Lₕ)-(acylamino acid) group, wherein —(Lₕ)— is an acylamino acid linker having an acylamino acid linker length of 1 to 7;

R^{3′} is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl, —(C₁-C₆)alkoxy(C₁-C₆)alkyl, —(C₁-C₆)alkoxy(C₁-C₆)alkenyl; or —(CH₂)ₙR⁸ where R⁸ is H, —CONH₂, —NR⁹R¹⁰, —CN or phenyl where R⁹ and R¹⁰ are independently hydrogen, —CF₃, phenyl, —(C₁-C₄)alkyl, —(C₁-C₄)alkylphenyl or -phenyl(C₁-C₄)alkyl and n is 1 to 8; and

R⁴ is H, —(C₃-C₁₄)alkyl, —(C₃-C₁₄)cycloalkyl, pyridyl, phenyl or phenyl substituted with from 1-5 substituents selected from the group consisting of —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy, —OR⁹; where R⁹ is hydrogen, —CF₃, phenyl, —(C₁-C₄)alkyl, —(C₁-C₄)alkylphenyl or -phenyl(C₁-C₄)alkyl; tetrazole; tetrazole substituted with —(C₁-C₄)alkyl or —(C₁-C₄)alkylphenyl: or naphthyl;

or a pharmaceutically acceptable racemate, solvate, tautomer, optical isomer, prodrug derivative or salt, thereof, comprising the steps of:

a) esterifying a compound of formula XVI

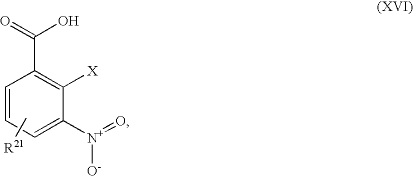

where X is halo;

to form a compound of formula XV

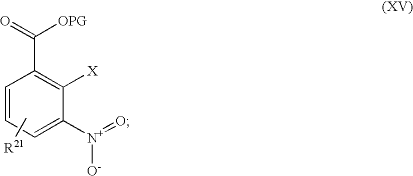

where PG is an acid protecting group

b) reducing a compound of formula XV to form a compound of formula XIV

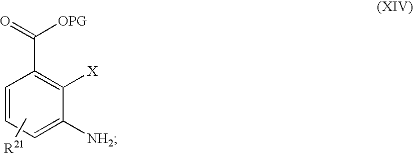

where PG is an acid protecting group

c) condensing a compound of formula XIV with a compound of formula XIII

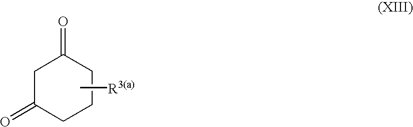

where R³ (a) is H, —O(C₁-C₄)alkyl, halo, —(C₁-C₆)alkyl, phenyl, —(C₁-C₄)alkylphenyl; phenyl substituted with —(C₁-C₆) alkyl, halo or —CF₃; —CH₂OSi (C₁-C₆) alkyl, furyl, thiophenyl, —(C₁-C₆)hydroxyalkyl, —(C₁-C₆) alkoxy(C₁-C₆) alkyl, —(C₁-C₆) alkoxy(C₁-C₆) alkenyl; or —(CH₂)ₙR⁸ where R⁸ is H, —NR⁹R¹⁰, —CN or phenyl where R⁹ and R¹⁰ are independently hydrogen, —CF₃, phenyl, —(C₁-C₄)alkyl, —(C₁-C₄)alkylphenyl or -phenyl(C₁-C₄)alkyl and n is 1 to 8; to form a compound of formula XII

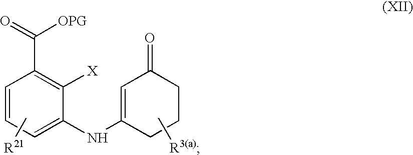

d) cyclizing a compound of formula XII to form a compound of formula XI

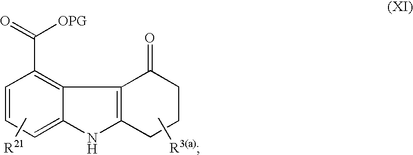

e) alkylating a compound of formula XI with an alkylating agent of the formula XCH₂R⁴, where X is halo to form a compound of formula X

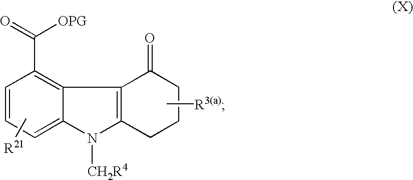

f) dehydrogenating a compound of formula X to form a compound of formula IX

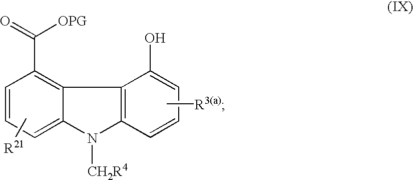

g) aminating a compound of formula IX to form a compound of formula VIII

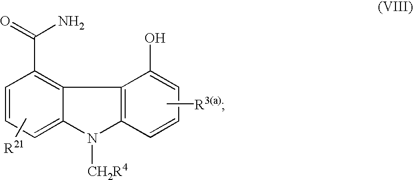

h) alkylating a compound of formula VIII with an alkylating agent of formula XCH₂R¹⁵ where X is halo and R¹⁵ is —CO₂R¹⁶, —SO₃R¹⁶, —P(O)(OR¹⁶)₂, or —P(O)(OR¹⁶)H, where R¹⁶ is an acid protecting group to form a compound of formula VII

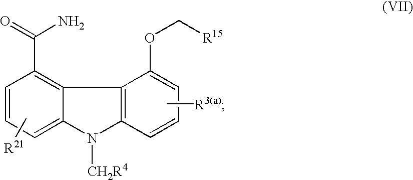

i) hydrolyzing a compound of formula VII to form the free acid thereof, and

j) coupling the free acid compound of step (I) with a C-terminal protected amino acid to from a compound of formula I.

This invention provides, in addition, a process for preparing compounds of formula II or a pharmaceutically acceptable racemate, solvate, tautomer, optical isomer, prodrug derivative or salt, thereof; which process comprises the steps of:

a) esterifying a compound of formula XVI

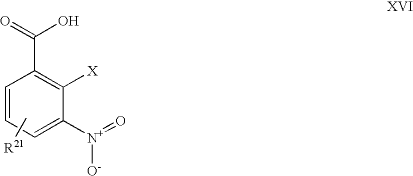

where X is halo to form a compound of formula XV

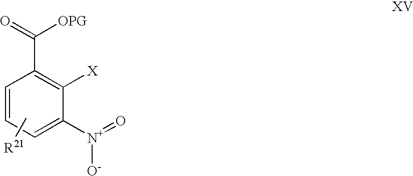

where PG is an acid protecting group;

b) condensing a compound of formula XV with a compound of formula XVII

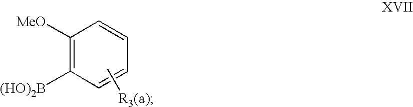

to form a compound of formula XVIII

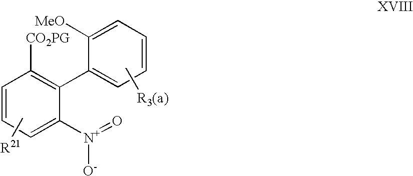

c) cyclizing a compound of formula XVIII to form a compound of formula XIX.

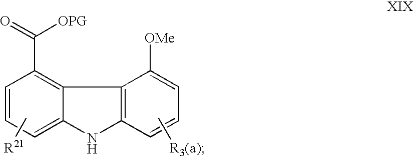

d) alkylating a compound of formula XIX with an alkylating agent of the formula XCH₂R⁴, where X is halo, to form a compound of formula XX

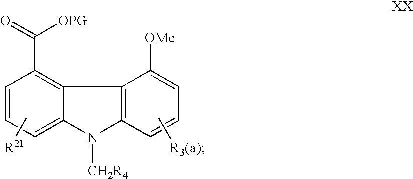

e) dealkylating a compound of formula XX to form a compound of formula IX

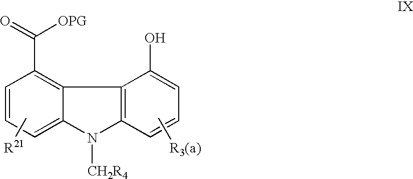

f) aminating compound of formula IX to form a compound of formula VIII

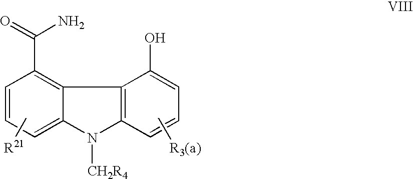

g) alkylating a compound of formula VIII with an alkylating agent of formula XCH₂R¹⁵, where X is halo and R¹⁵ is —CO₂R¹⁶, where R¹⁶ is an acid protecting group to form a compound of formula VII

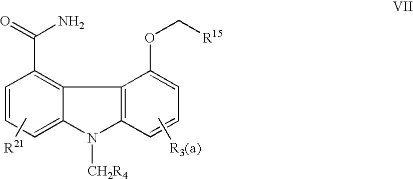

h) optionally hydrolyzing a compound of formula VII to form a free acid compound thereof, and optionally salifying the free acid; and

i) converting a compound of formula VII or the free acid thereof to form a compound of formula II.

Other objects, features and advantages of the present invention will become apparent from the subsequent description and the appended claims.

### DETAILED DESCRIPTION OF THE INVENTION

Definitions:

As used herein, the term, “alkyl” by itself or as part of another substituent means, unless otherwise defined, a straight or branched chain monovalent hydrocarbon radical such as methyl, ethyl, n-propyl, isopropyl, n-butyl, tertiary butyl, isobutyl, sec-butyl tert butyl, n-pentyl, isopentyl, neopentyl, heptyl, hexyl, octyl, nonyl, decyl, undecyl, dodecyl, tridecyl, tetradecyl and the like. The term “alkyll” includes —(C₁-C₂)alkyl, —(C₁-C₄)alkyl, —(C₁-C₆)alkyl, —(C₅-C₁₄)alkyl, and —(C₁-C₁₀)alkyl.

The term “alkenyl” as used herein represents an olefinically unsaturated branched or linear group having at least one double bond. Examples of such groups include radicals such as vinyl, allyl, 2-butenyl, 3-butenyl, 2-pentenyl, 3-pentenyl, 4-pentenyl, 2-hexenyl, 3-hexenyl, 4-hexenyl, 5-hexenyl, 2-heptenyl, 3-heptenyl, 4-heptenyl, 5-heptenyl, 6-heptenyl as well as dienes and trienes of straight and branched chains.

The term “alkynyl” denotes such radicals as ethynyl, propynyl, butynyl, pentynyl, hexynyl, heptynyl as well as di- and tri-ynes.

The term “halo” means chloro, fluoro, bromo or iodo.

The term “—(C₁-C₄)alkoxy”, as used herein, denotes a group such as methoxy, ethoxy, n-propoxy, isopropoxy, n-butoxy, t-butoxy and like groups, attached to the remainder of the molecule by the oxygen atom.

The term “phenyl(C₁-C₄)alkyl” refers to a straight or branched chain alkyl group having from one to four carbon atoms attached to a phenyl ring which chain is attached to the remainder of the molecule. Typical phenylalkyl groups include benzyl, phenylethyl, phenylpropyl, phenylisopropyl, and phenylbutyl.

The term “—(C₁-C₄)alkylthio” defines a straight or branched alkyl chain having one to four carbon atoms attached to the remainder of the molecule by a sulfur atom. Typical —(C₁-C₄)alkylthio groups include methylthio, ethylthio, propylthio, butylthio and the like.

The term “—(C₃-C₁₄)cycloalkyl” includes groups such as cyclopropyl, cyclobutyl, cyclopentyl, cyclohexyl, cycloheptyl, cyclooctyl, cyclononyl, cyclodecyl, cycloundecyl, cyclododecyl, cyclotridecyl, cyclotetradecyl and the like. The term “—(C₃-C₁₄)cycloalkyl” includes and —(C₃-C₇)cycloalkyl. The term, “heterocyclic radical”, refers to radicals derived from monocyclic or polycyclic, saturated or unsaturated, substituted or unsubstituted heterocyclic nuclei having 5 to 14 ring atoms and containing from 1 to 3 hetero atoms selected from the group consisting of nitrogen, oxygen or sulfur. Typical heterocyclic radicals are pyridyl, thienyl, fluorenyl, pyrrolyl, furanyl, thiophenyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, thianaphtheneyl, dibenzothiophenyl, indazolyl, imidazo (1.2-A)pyridinyl, benzotriazolyl, anthranilyl, 1,2-benzisoxazolyl, benzoxazolyl, benzothiazolyl, purinyl, pryidinyl, dipyridylyl, phenylpyridinyl, benzylpyridinyl, pyrimidinyl, phenylpyrimidinyl, pyrazinyl, 1,3,5-triazinyl, quinolinyl, phthalazinyl, quinazolinyl, and quinoxalinyl.

The term “carbocyclic radical” refers to radicals derived from a saturated or unsaturated, substituted or unsubstituted 5 to 14 membered organic nucleus whose ring forming atoms (other than hydrogen) are solely carbon atoms. Typical carbocyclic radicals are cycloalkyl, cycloalkenyl, phenyl, naphthyl, norbornanyl, bicycloheptadienyl, tolulyl, xylenyl, indenyl, stilbenyl, terphenylyl, diphenylethylenyl, phenylcyclohexeyl, acenaphthylenyl, and anthracenyl, biphenyl, bibenzylyl and related bibenzylyl homologues represented by the formula (bb),

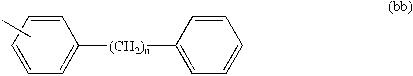

where n is an integer from 1 to 8.

The term, “non-interfering substituent”, refers to radicals suitable for substitution at positions 1, 2, 3, 7 and/or 8 on the tricyclic nucleus (as depicted in Formula III) and radical(s) suitable for substitution on the heterocyclic radical and carbocyclic radical as defined above. Illustrative non-interfering radicals are hydrogen, —(C₁-C₁₄)alkyl, —(C₂-C₆)alkenyl, —(C₂-C₆)alkynyl, —(C₇-C₁₂)aralkyl, —(C₇-C₁₂)alkaryl, —(C₃-C₈)cycloalkyl, —(C₃-C₈)cycloalkenyl, phenyl, tolulyl, xylenyl, biphenyl, —(C₁-C₆)alkoxy, —(C₂-C₆)alkenyloxy, —(C₂-C₆)alkynyloxy, —(C₁-C₁₂)alkoxyalkyl, —(C₁-C₁₂)alkoxyalkyloxy, —(C₁-C₁₂)alkylcarbonyl, —(C₁-C₁₂)alkylcarbonylamino, —(C₁-C₁₂)alkoxyamino, —(C₁-C₁₂)alkoxyaminocarbonyl, —(C₁-C₁₂)alkylamino, —(C₁-C₆)alkylthio, —(C₁-C₁₂)alkylthiocarbonyl, —(C₁-C₆)alkylsulfinyl, —(C₁-C₆)alkylsulfonyl, —(C₁-C₆)haloalkoxy, —(C₁-C₆)haloalkylsulfonyl, —(C₁-C₆)haloalkyl, —(C₁-C₆)hydroxyalkyl, —(CH₂)ₙCN, —(CH₂)ₙNR⁹R¹⁰, —C(O)O(C₁-C₆ alkyl), —(CH₂)ₙO(C₁-C₆ alkyl), benzyloxy, phenoxy, phenylthio; —(CONHSO₂)R¹⁵ where R¹⁵ is —(C₁-C₆)alkyl; —CF₃, naphthyl or —(CH₂)ₛphenyl where s is 0-5; —CHO, —CF₃, —OCF₃, pyridyl, amino, amidino, halo, carbamyl, carboxyl, carbalkoxy, —(CH₂)ₙCO₂H, cyano, cyanoguanidinyl, guanidino, hydrazide, hydrazino, hydrazido, hydroxy, hydroxyamino, nitro, phosphono, —SO₃H, thioacetal, thiocarbonyl, furyl, thiophenyl —COR⁹, —CONR⁹R¹⁰, —NR⁹R¹⁰, —NCHCOR⁹, —SO₂R⁹, —OR⁹, —SR⁹, CH₂SO₂R⁹, tetrazolyl or tetrazolyl substituted with —(C₁-C₆)alkyl, phenyl or —(C₃-C₄)alkylphenyl, —(CH₂)ₙOSi(C₁-C₆)alkyl and (C₁-C₆)alkylcarbonyl; where n is from 1 to 8 and R⁹ and R¹⁰ are independently hydrogen, —CF₃, phenyl, —(C₁-C₄) alkyl, —(C₁-C₄) alkylphenyl or -phenyl (C₁-C₄) alkyl

The term, “organic substituent” refers to a monovalent radical consisting of carbon and hydrogen with or without oxygen, nitrogen, sulfur, halogen, or other elements. Illustrative organic substituents are C₁-C₈ alkyl, aryl, C₇-C₁₄ aralkyl, C₇-C₁₄ alkaryl, C₃-C₈ cycloalkyl, C₁-C₈ alkoxyalkyl and these groups substituted with halogen, —CF₃, —OH, C₁-C₈ alkyl, amino, carbonyl, and —CN.

The term “acylamino acid” is a group represented by the formula:

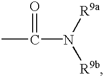

wherein R^{9a} is selected from the group consisting of H, (C₁-C₆)alkyl, (C₁-C₆)alkoxy, heteroaryl and aryl, —CF₃; and wherein NR^{9b} is an amino acid residue of either a natural or unnatural amino acid with the nitrogen atom being part of the amino group of the amino acid. A typical amino acid is selected from the group comprising isoleucine, valine, phenylalanine, aspartic acid, leucine, glycine, asparagine, cystein, glutamine, glutamic acid, histidine, lysine, methionine, serine, threonine, tryptophan, tyrosine and derivatives thereof. Also contemplated within the definition of amino acid is 1-proline, d-proline and derivatives thereof. Also contemplated within the definition of amino acids are peptides, polypeptides and derivatives thereof.

The term “substituted group” is an organic group substituted with one or more non-interfering substituents.

The term C-terminal protected amino acid is an amino acid group wherein the carboxy terminal is protected by an acid protecting group known in the art to form groups such as the ester or amide, e.t.c., while the amino terminal is available for coupling with substrate carboxy groups by peptide coupling methodologies known to one of skill in the art.

The terms “linker” and “acylamino acid linker” as used herein are synonymous.

The phrase, “amino acid residue” refers to the portion of the amino acid group coupled at the nitrogen atom of the amino terminus. It is the amino acid less a hydrogen atom from the amino terminus. It is further illustrated as used herein for the amino acid alanine attached at the nitrogen atom as shown below:

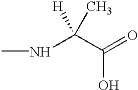

The phrase, “acylamino acid linker” refers to a divalent linking group symbolized as, —(Lₕ)—, which has the function of joining the 5-position of the tricyclic nucleus to an acylamino acid group in the general relationship:

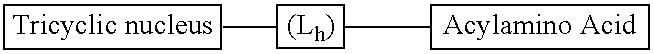

The words, “acylamino acid linker length”, refer to the number of atoms (excluding hydrogen) in the shortest chain of the linking group —(Lₕ)— that connects the 5-position of the tricyclic nucleus with the acylamino acid group. The presence of a carbocyclic ring in —(Lₕ)— counts as the number of atoms approximately equivalent to the calculated diameter of the carbocyclic ring. Thus, a benzene or cyclohexane ring in the acid linker counts as 2 atoms in calculating the length of —(Lₕ)—.

Illustrative acylamino acid linker groups are;

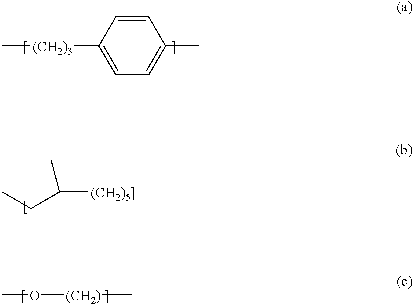

wherein, groups (a), (b) and (c) have acylamino acid linker lengths of 5, 7, and 2, respectively;

The term, “acidic group” means an organic group which when attached to a tricyclic nucleus, through suitable linking atoms (hereinafter defined as the “acid linker”), acts as a proton donor capable of hydrogen bonding. Illustrative of an acidic group are the following:

—CO₂H,

-5-tetrazolyl,

—SO₃H,

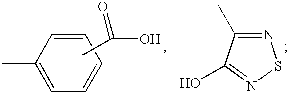

The salts of the above tricyclic compounds are an additional aspect of the present invention. In those instances where the compounds of the invention possess acidic functional groups various salts may be formed which are more water soluble and physiologically suitable than the parent compound. Representative pharmaceutically acceptable salts include but are not limited to the alkali and alkaline earth salts such as lithium, sodium, potassium, calcium, magnesium, aluminum and the like. Salts are conveniently prepared from the free acid by treating the acid in solution with a base or by exposing the acid to an ion exchange resin. Included within the definition of pharmaceutically acceptable salts are the relatively non-toxic, inorganic and organic base addition salts of compounds of the present invention, for example, ammonium, quaternary ammonium, and amine cations, derived from nitrogenous bases of sufficient basicity to form salts with the compounds of this invention (see, for example, S. M. Berge, et al., “Pharmaceutical Salts,” J. Phar. Sci., 66: 1-19 (1977)).

Compounds of the invention may have chiral centers and exist in optically active forms. R- and S-isomers and racemic mixtures are contemplated by this invention. A particular stereoisomer may be prepared by known methods using stereospecific reactions with starting materials containing asymmetric centers already resolved or, alternatively, by subsequent resolution of mixtures of stereoisomers using known methods.

Prodrugs are derivatives of the compounds of the invention which have chemically or metabolically cleavable groups and become by solvolysis or under physiological conditions the compounds of the invention which are pharmaceutically active in vivo. Derivatives of the compounds of this invention have activity in both their acid and base derivative forms, but the acid derivative form often offers advantages of solubility, tissue compatibility, or delayed release in a mammalian organism (see, Bundgard, H., Design of Prodrugs, pp. 7-9, 21-24, Elsevier, Amsterdam 1985). Prodrugs include acid derivatives, such as, esters prepared by reaction of the parent acidic compound with a suitable alcohol, or amides prepared by reaction of the parent acid compound with a suitable amine. Simple aliphatic esters (e.g., methyl, ethyl, propyl, isopropyl, butyl, sec-butyl, tert-butyl) or aromatic esters derived from acidic groups pendent on the compounds of this invention are preferred prodrugs. Other preferred esters include morpholinoethyloxy, diethylglycolamide and diethylaminocarbonylmethoxy. In some cases it is desirable to prepare double ester type prodrugs such as (acyloxy) alkyl esters or ((alkoxycarbonyl)oxy)alkyl esters.

The term “acid protecting group” is used herein as it is frequently used in synthetic organic chemistry, to refer to a group which will prevent an acid group from participating in a reaction carried out on some other functional group in the molecule, but which can be removed when it is desired to do so. Such groups are discussed by T. W. Greene in chapter 5 of Protective Groups in Organic Synthesis, John Wiley and Sons, New York, 1981, incorporated herein by reference in its entirety.

Examples of acid protecting groups include ester or amide derivatives of the acid group, such as, methyl, methoxymethyl, methyl-thiomethyl, tetrahydropyranyl, methoxyethoxymethyl, benzyloxymethyl, phenyl, aryl, ethyl, 2,2,2-trichloroethyl, 2-methylthioethyl, t-butyl, cyclopentyl, triphenylmethyl, diphenylmethyl, benzyl, trimethylsilyl, N,N-dimethyl, pyrrolidinyl, piperidinyl, or o-nitroanilide. A preferred acid-protecting group is methyl.

Preferred Compounds of the Invention

Preferred Subgroups of Compounds of Formula (I):

A preferred subclass of compounds of formula (I) are those wherein R²¹ is selected from the group hydrogen, halo, —(C₁-C₃)alkyl, —(C₃-C₄)cycloalkyl, —(C₃-C₄)cycloalkenyl, —O(C₁-C₂)alkyl and —S(C₁-C₂)alkyl. Another preferred subclass of compounds of formula (I) are those wherein for R²′, —(Lₕ)— is an alkyl chain of 1 or 2 carbon atoms.

Another preferred subclass of compounds of formula (I) are those wherein for R⁴, group R⁸⁰ is selected from the group consisting of cycloalkyl, cycloalkenyl, phenyl, naphthyl, norbornanyl, bicycloheptadienyl, tolulyl, xylenyl, indenyl, stilbenyl, terphenylyl, diphenylethylenyl, phenyl-cyclohexenyl, acenaphthylenyl, and anthracenyl, biphenyl, bibenzylyl and related bibenzylyl homologues represented by the formula (bb),

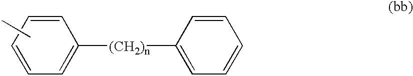

where n is a number from 1 to 8. Particularly preferred are compounds wherein R⁴ is selected from the group consisting of

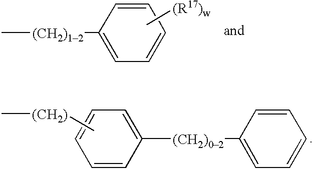

where R¹⁷ is a radical independently selected from halo, —(C₁-C₁₀)alkyl, —(C₁-C₁₀)alkoxy, —S—(C₁-C₁₀ alkyl), and —(C₁-C₁₀)haloalkyl, and w is a number from 0 to 5.

Another preferred subclass of compounds of formula (I) are those wherein R² is a substituent having an acylamino acid linker with an acylamino acid linker length of 2 or 3 and the acylamino acid linker group, —(Lₕ)—, for R^{2′}, is selected from a group represented by the formula;

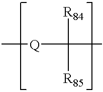

where Q is selected from the group —(CH₂)—, —O—, —NH—, and —S—, and R₈₄ and R₈₅ are each independently selected from hydrogen, —(C₁-C₁₀)alkyl, aryl, —(C₁-C₁₀)alkylaryl, -aryl(C₁-C₁₀)alkyl, carboxy, carbalkoxy, and halo. Most preferred are compounds where the acylamino acid linker, —(Lₕ)—, for R², is selected from the specific groups;

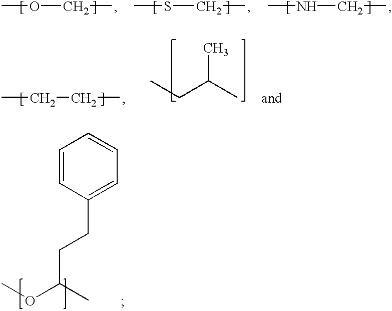

Another preferred subclass of compounds of formula (I) are those wherein R^{2′} is a substituent having an acylamino acid linker with an acylamino acid linker length of 3 to 8 atoms and the acid linker group, —(Lₕ)—, for R^{2′} is selected from;

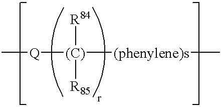

where r is a number from 1 to 7, s is 0 or 1, and Q is selected from the group —(CH₂)—, —O—, —NH—, and —S—, and R₈₄ and R₈₅ are each independently selected from hydrogen, —(C₁-C₁₀)alkyl, aryl, —(C₁-C₁₀)alkylaryl, -aryl(C₁-C₁₀)alkyl, carboxy, carbalkoxy, and halo. Most preferred are compounds where the acylamino acid linker, —(Lₕ)—, for R^{2′} is selected from the specific groups;

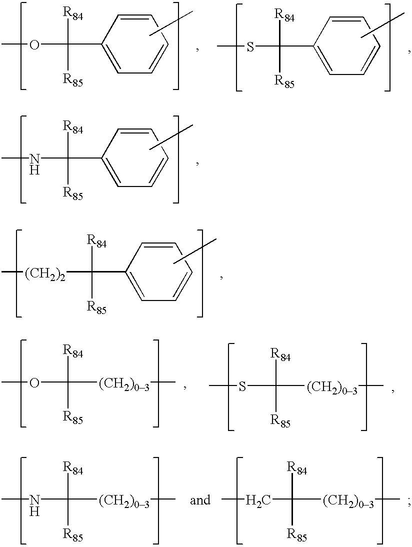

wherein R₈₄ and R₈₅ are each independently selected from hydrogen, —(C₁-C₁₀)alkyl, aryl, —(C₁-C₁₀)alkaryl, —(C₁-C₁₀)aralkyl, carboxy, carbalkoxy, and halo.

Another preferred subclass of compounds of formula (I) are those wherein R^{3′} is selected from hydrogen and non-interfering substituents, with the non-interfering substituents being selected from the group consisting of hydrogen, —(C₁-C₆)alkyl, —(C₂-C₆)alkenyl, —(C₂-C₆)alkynyl, —(C₇-C₁₂)aralkyl, —(C₇-C₁₂)alkaryl, —(C₃-C₈)cycloalkyl, —(C₃-C₈)cycloalkenyl, phenyl, tolulyl, xylenyl, biphenyl, —(C₁-C₆)alkoxy, —(C₂-C₆)alkenyloxy —(C₂-C₆)alkynyloxy, —(C₁-C₁₂)alkoxyalkyl, —(C₁-C₁₂)alkoxyalkyloxy, —(C₁-C₁₂)alkylcarbonyl, —(C₁-C₁₂)alkylcarbonylamino, —(C₁-C₁₂)alkoxyamino, —(C₁-C₁₂)alkoxyaminocarbonyl, —(C₁-C₁₂)alkylamino, —(C₁-C₆)alkylthio, —(C₁-C₁₂)alkylthiocarbonyl, —(C₁-C₆)alkylsulfinyl, —(C₁-C₆)alkylsulfonyl, —(C₁-C₆)haloalkoxy, —(C₁-C₆)haloalkylsulfonyl, —(C₁-C₆)haloalkyl, —(C₁-C₆)hydroxyalkyl, —C(O)O(C₁-C₆ alkyl), —(CH₂)ₙO(C₁-C₆ alkyl), benzyloxy, halo, phenylthio; phenyl substituted with —(C₁-C₆)alkyl, halo, or —CF₃; furyl, thiophenyl, —(CH₂)ₙCN, —(CH₂)ₙR⁸ where R⁸ is H, —CONH₂, —NR⁹R¹⁰, —CN or phenyl where R⁹ and R¹⁰ are independently Hydrogen, —(C₁-C4) alkyl or -phenyl(C₁-C₄); —CHO, amino, amidino, carbamyl, carboxyl, carbalkoxy, —(CH₂)ₙCO₂H, cyano, cyanoguanidinyl, guanidino, hydrazide, hydrazino, hydrazido, hydroxy, hydroxyamino, nitro, phosphono, —SO₃H, thioacetal, thiocarbonyl, and —(C₁-C6)alkylcarbonyl; where n is from 1 to 8.

Another preferred group of substituents for R^{3′} include H, —O(C₁-C₄)alkyl, halo, —(C₁-C₆)alkyl, phenyl, —(C₁-C₆)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙR⁸ where R⁸ is H, —CONH₂, —NR⁹R¹⁰, —CN or phenyl where R⁹ and R¹⁰ are independently hydrogen, —(C₁-C₄)alkyl or -phenyl(C₁-C₄)alkyl and n is 1 to 8;

Yet another preferred group for R^{3′}, include H, —O(C₁-C₄)alkyl, halo, —(C₁-C₆)alkyl, phenyl, —(C₁-C₄)alkylphenyl; phenyl substituted with —(C₁-C₆)alkyl, halo or —CF₃; —CH₂OSi(C₁-C₆)alkyl, furyl, thiophenyl, —(C₁-C₆)hydroxyalkyl; or —(CH₂)ₙR⁸ where R⁸ is H, —NR⁹R¹⁰, —CN or phenyl where R⁹ and R¹⁰ are independently —(C₁-C₄)alkyl or -phenyl(C₁-C₄)alkyl and n is 1 to 8.

Preferred compounds of the invention are those having the general formula (IIa)

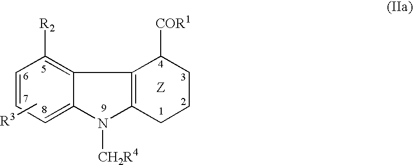

wherein;

R¹ is —CONH₂, —NHNH₂, or —NH₂;

R² is selected from the group consisting of —O(CH₂)ₘR^{5′},

where

R^{5′} is —CONR⁹R¹⁰, where R⁹ and R¹⁰ are independently —(C₁-C₆)alkyl or —CF₃; phenyl or phenyl substituted with —(C₁-C₆)alkyl; and —(Lₕ)-(acylamino acid) is a group wherein —(Lₕ)— is an acylamino acid linker having an acylamino acid linker length of 1 to 7 and m is 1-2; R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙR⁸ where R⁸ is H, —CONH₂, NR⁹R¹⁰, —CN or phenyl where R⁹ and R¹⁰ are independently —(C₁-C₄)alkyl or -phenyl(C₁-C₄)alkyl and n is 1 to 8;

R⁴ is H, —(C₁-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl(C₁-C₄) alkyl, —(C₁-C₄)alkylphenyl, phenyl, phenoxy or naphthyl;

Z is cyclohexenyl, or phenyl;

or a pharmaceutically acceptable racemate, solvate, tautomer, optical isomer, prodrug derivative or salt, thereof.

Preferred substituents of compounds of formula I, II and IIa include the following:

(a) R¹ is —CONH₂, —NH₂, —NHNH_{2;}

(b) R¹ is —CONH₂;

(c) R^{2′} is —O(CH₂)ₘR⁵ where R⁵ is an acylamino acid group represented by the formula:

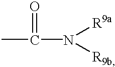

wherein R^{9a} is selected from the group consisting of H,. (C₁-C₆)alkyl, (C₁-C₆)alkoxy, heteroaryl and aryl, —CF₃; and wherein NR^{9b} is an “amino acid residue” of either a natural or unnatural amino acid with the nitrogen atom being part of the amino group of the amino acid. An amino acid residue is selected from the group comprising isoleucine, valine, phenylalanine, aspartic acid, leucine, glycine, asparagine, cystein, glutamine, glutamic acid, histidine, lysine, methionine, serine, threonine, tryptophan, 1-proline, d-proline, tyrosine and derivatives thereof.

(g) R³ is —H, —O(C₁-C₄ alkyl) or —(CH₂)ₙR⁸ where n=2 and

R⁸ is H or phenyl;

(h) R³ is H, or —O(C₁-C₄ alkyl);

(i) R³ is —(CH₂)ₙR⁸ where R⁸ is —(C₁-C₄)alkyl;

(j) R⁴ is phenyl;

(k) R⁴ is phenyl substituted at the 2- and 6-position of the phenyl ring with —(C₁-C₄)alkyl, (C₁-C₄)alkoxy, halo or phenyl;

(l) R⁴ is phenyl substituted at the 2- or 6-position of the phenyl ring with —(C₁-C₄)alkyl, —(C₁-C₄)alkoxy, halo or phenyl;

(m) R⁴ is phenyl substituted at the 3- or 5-position of the phenyl ring with —(C₁-C₄)alkyl, —(C₁-C₄)alkoxy, halo or phenyl;

(n) R⁴ is —(C₆-C₁₄)alkyl or —(C₆-C₁₄)cycloalkyl;

(o) Z is cyclohexenyl or phenyl;

(p) R⁵ is —CO₂H, —CO₂(C₁-C₄ alkyl), —NHSO₂(C₁-C₆)alkyl, —CONHSO₂(C₁-C₆)alkyl, tetrazolyl, phenyl, or phenyl substituted with —CO₂H or —CO₂(C₁-C₄ alkyl) where R⁶ and R⁷ are each independently —OH or —O(C₁-C₄ alkyl) and m is 1-3;

(q) R⁵ is —CO₂H, —CO₂(C₁-C₄ alkyl), phenyl, or phenyl substituted with —CO₂H or —CO₂(C₁-C₄ alkyl) where R⁶ and R⁷ are each independently —OH or —O(C₁-C₄ alkyl) and m is 1-3;

(r) Z is cyclohexenyl;

(s) Z is phenyl; and

(u) R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆) alkyl, furyl, thiophenyl, —(C₁-C₆)hydroxyalkyl —(C₁-C₆)alkoxy(C₁-C₆) alkyl, —(C₁-C₆)alkoxy(C₁-C₆)alkenyl, —(C₁-C₆)alkoxy(C₁-C₆)alkyl, —(C₁-C₆)alkoxy, —(C₁-C₆)alkenyl; or —(CH₂)ₙR⁸ where R⁸ is H, —CONH₂, —NR⁹R¹⁰, —CN or phenyl where R⁹ and R¹⁰ are independently hydrogen, —CF₃, phenyl, —(C₁-C₄)alkyl, —(C₁-C₄)alkylphenyl or -phenyl(C₁-C₄)alkyl and n is 1 to 8.

Also preferred for formula I is a compound where R⁴ is represented by —(CH₂)m-R¹² wherein m is an integer from 1 to 6, and R¹² is (d) a group represented by the formula:

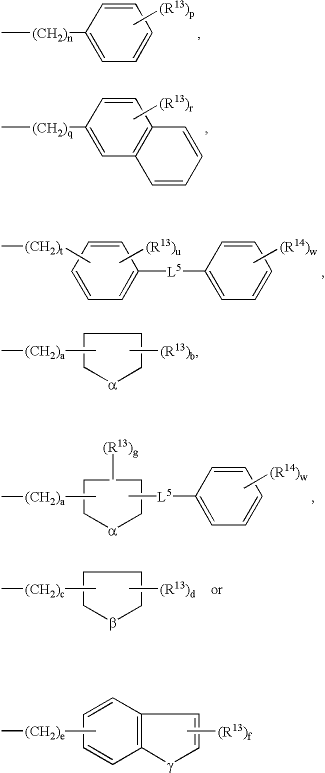

wherein a, c, e, n, q, and t are independently an integer from 0 to 2, R¹³ and R¹⁴ are independently selected from a halogen, C₁ to C₈ alkyl, C₁ to C₈ alkyloxy, C₁ to C₈ alkylthio, aryl, heteroaryl, and C₁ to C₈ haloalkyl, α is an oxygen atom or a sulfur atom, L⁵ is a bond, —(CH₂)v-,

—C═C—, —CC—, —O—, or —S—, v is an integer from 0 to 2, β is —CH₂— or —(CH₂)₂—, γ is an oxygen atom or a sulfur atom, b is an integer from 0 to 3, d is an integer from 0 to 4, f, p, and w are independently an integer from 0 to 5, r is an integer from 0 to 7, and u is an integer from 0 to 4, or is (e) a member of (d) substituted with at least one substituent selected from the group consisting of C₁ to C₆ alkyl, C₁ to C₈ alkyloxy, C₁ to C₈ haloalkyloxy, C₁ to C₈ haloalkyl, aryl, and a halogen.

Carbazole and Tetrahydrocarbazole sPLA₂ Inhibitors

Carbazole and tetrahydrocarbazole sPLA₂ inhibitors and methods of making these compounds are set out in U.S. patent application Ser. No. 09/063,066 filed Apr. 21, 1998 (titled, “Substituted Carbazoles and 1,2,3,4-Tetrahydrocarbazoles”), the entire disclosure of which is incorporated herein by reference. The method of the invention includes treatment of a mammal with these compounds.

The method of the invention is for treatment of a mammal, including a human, afflicted with inflammation, said method comprising administering to said human a therapeutically effective amount of carbazole or tetrahydrocarbazole represented by the following:

A compound of the formula (Ie)

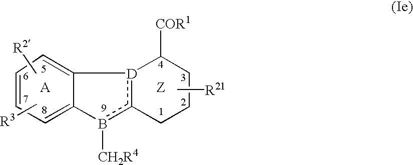

wherein;

A is phenyl or pyridyl wherein the nitrogen is at the 5-, 6-, 7- or 8-position; one of B or D is nitrogen and the other is carbon;

Z is cyclohexenyl, phenyl, pyridyl, wherein the nitrogen is at the 1-, 2-, or 3-position, or a 6-membered heterocyclic ring having one heteroatom selected from the group consisting of sulfur or oxygen at the 1-, 2- or 3-position, and nitrogen at the 1-, 2-, 3- or 4-position;

[symbol] is a double or single bond;

R⁴ is selected from groups (a), (b) and (c) where;

(a) is —(C₅-C₂₀)alkyl, —(C₅-C₂₀), —(C₅-C₂₀)alkynyl, carbocyclic radicals, or heterocyclic radicals, or

(b) is a member of (a) substituted with one or more independently selected non-interfering substituents; or

(c) is the group —(L)—R⁸⁰; where, —(L)— is a divalent linking group of 1 to 12 atoms selected from carbon, hydrogen, oxygen, nitrogen, and sulfur; wherein the combination of atoms in —(L)— are selected from the group consisting of (i) carbon and hydrogen only, (ii) one sulfur only, (iii) one oxygen only, (iv) one or two nitrogen and hydrogen only, (v) carbon, hydrogen, and one sulfur only, and (vi) and carbon, hydrogen, and oxygen only; and where R⁸⁰ is a group selected from (a) or (b);

R²¹ is a non-interfering substituent;

R^{1′} is —NHNH₂, —NH₂ or —CONH₂;

R^{2′} is the group —O(CH₂)ₜR^{5′} where

R^{5′}, is

(a) —CONR⁹R¹⁰, where R⁹ and R¹⁰ are independently Hydrogen, —(C₁-C₆)alkyl, —(C₁-C₆)alkyl substituted with —CO₂H or —CO₂(C₁-C₄)alkyl or —CF₃; phenyl or phenyl substituted with —(C₁-C₆)alkyl; or

(b) the group —(Lₕ)-(acylamino acid) group, wherein —(Lₕ)— is an acid linker having an acid linker length of 1 to 7, t is 1-5; and the acylamino acid group is the group —C(O)NR^{9a}R^{9b}, wherein R^{9a} is H, —(C₁-C₆)alkyl or —CF₃; phenyl or phenyl substituted with —CO₂H or —CO₂(C₁-C₄)alkyl and NR^{9b} is an “amino acid residue”;

R^{3′} is selected from non-interfering substituent, carbocyclic radicals, carbocyclic radicals substituted with non-interfering substituents, heterocyclic radicals, and heterocyclic radicals substituted with non-interfering substituents; or a pharmaceutically acceptable racemate, solvate, tautomer, optical isomer, prodrug derivative or salt thereof;

provided that; when R^{3′} is H, R⁴ is benzyl and m is 1 or 2; R^{2′}, cannot be —O(CH₂)ₘH; and

provided that when d is nitrogen, the heteroatom of z is selected from the group consisting of sulfur or oxygen at the 1-, 2- or 3-position and nitrogen at the 1-, 2-, 3- or 4-position.

Preferred in the practice of the method of the invention are compounds represented by the formula (IIe):

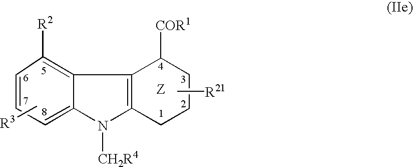

wherein;

Z is cyclohexenyl, or phenyl;

R²¹ is a non-interfering substituent;

R¹ is —NHNH₂, —CONH₂ OR —NH₂;

R² is the group —O(CH₂)_{M}R⁵ where

R⁵ is -(acylamino acid) where the acylamino acid group is represented by the formula

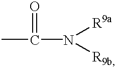

wherein R^{9a} is selected from the group consisting of H, (C₁-C₆)alkyl, (C₁-C₆)alkoxy, heteroaryl and aryl, —CF₃; and wherein NR^{9b} is an amino acid residue of either a natural or unnatural amino acid with the nitrogen atom being part of the amino group of the amino acid, and where m is 1-3;

R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙR⁸ where R⁸ is H, —CONH₂, —NR⁹R¹⁰, —CN or phenyl where R⁹ and R¹⁰ are independently —(C₁-C₄)alkyl or -phenyl(C₁-C₄)alkyl and n is 1 to 8;

R⁴ is H, —(C₅-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy or naphthyl;

or a pharmaceutically acceptable racemate, solvate, tautomer, optical isomer, prodrug derivative or salt, thereof.

Preferred specific compounds including all salts and prodrug derivatives thereof, for practicing the method of the invention are as follows:

[[5-Carbamoyl-9-(phenylmethyl)carbazol-4-yl]oxy]acetamide

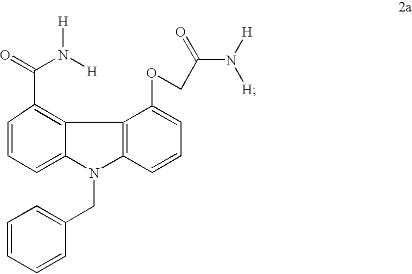

[[5-Carbamoyl-9-(phenylmethyl)carbazol-4-yl]oxy]-N-(ethyl)acetamide

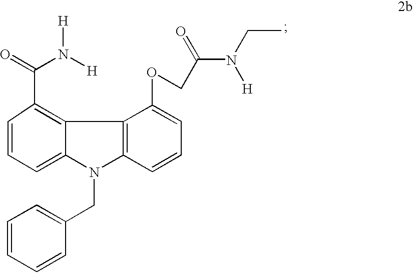

N-[[[5-Carbamoyl-9-(phenylmethyl)carbazol-4-yl]oxy]acetyl]glycine

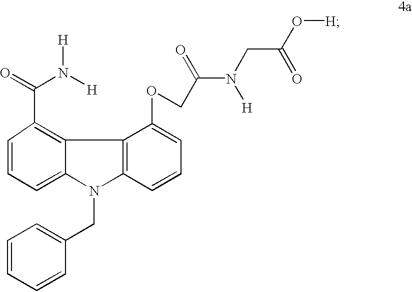

N-[[[5-Carbamoyl-9-(phenylmethyl)carbazol-4-yl]oxy]acetyl]glycine methyl ester

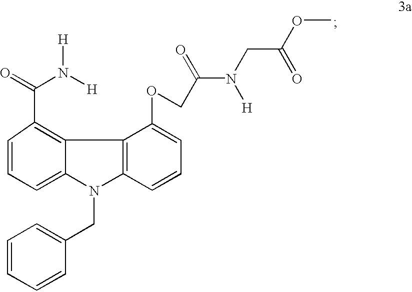

N-[[[5-Carbamoyl-9-(phenylmethyl)carbazol-4-yl]oxy]acetyl]glycine

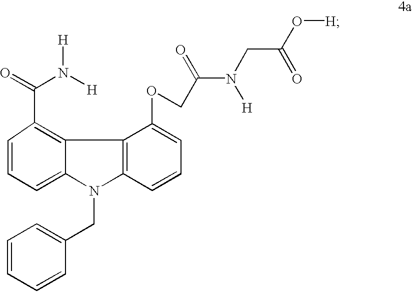

N-[[[5-Carbamoyl-9-(phenylmethyl)carbazol-4-yl]oxy]acetyl]-L-alanine

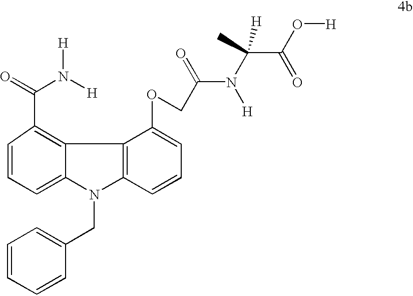

N-[[[5-Carbamoyl-9-(phenylmethyl)carbazol-4-yl]oxy]acetyl]-L-alanine methyl ester

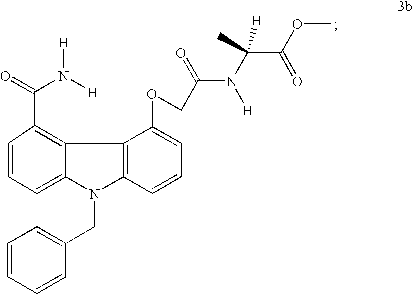

N-[[[5-Carbamoyl-9-(phenylmethyl)carbazol-4-yl]oxy]acetyl]-L-alanine

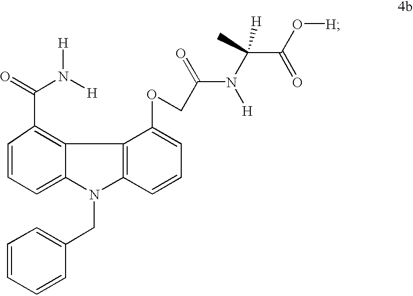

N-[[[5-Carbamoyl-9-(phenylmethyl)carbazol-4-yl]oxy]acetyl]-L-leucine

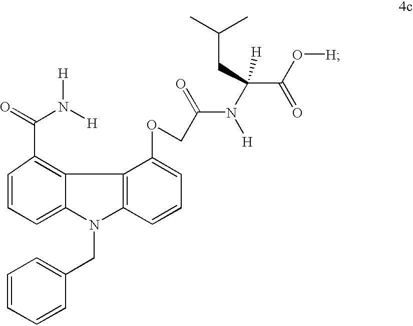

N-[[[5-Carbamoyl-9-(phenylmethyl)carbazol-4-yl]oxy]acetyl]-L-leucine methyl ester

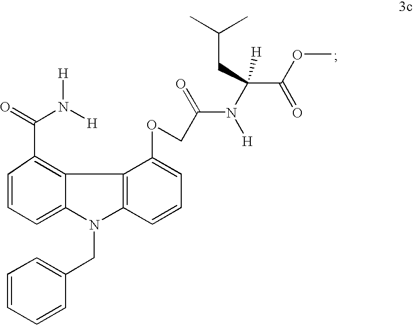

N-[[[5-Carbamoyl-9-(phenylmethyl)carbazol-4-yl]oxy]acetyl]-L-leucine

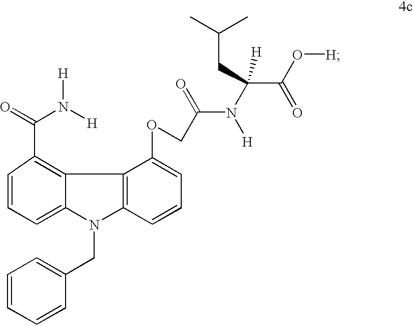

N-[[[5-Carbamoyl-9-(phenylmethyl)carbazol-4-yl]oxy]acetyl]-L-aspartic acid

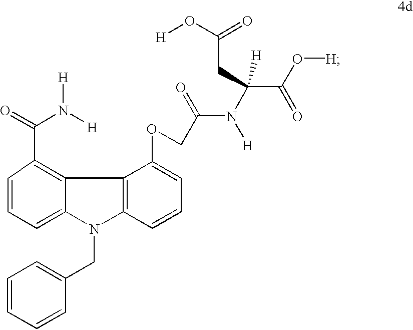

N-[[[5-Carbamoyl-9-(phenylmethyl)carbazol-4-yl]oxy]acetyl]-L-aspartic acid dimethyl ester

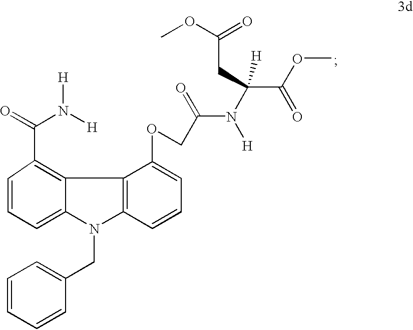

N-[[[5-Carbamoyl-9-(phenylmethyl)carbazol-4-yl]oxy]acetyl]-L-aspartic acid

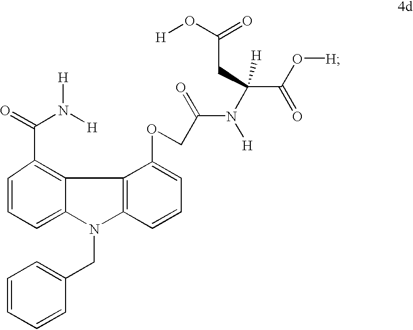

N-[[[5-Carbamoyl-9-(phenylmethyl)carbazol-4-yl]oxy]acetyl]-L-glutamic acid

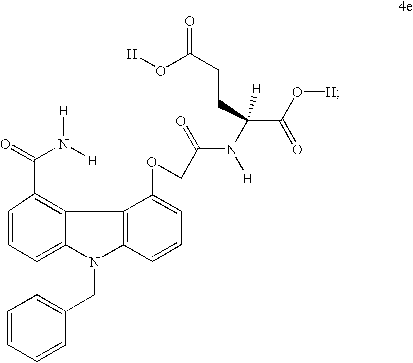

N-[[[5-Carbamoyl-9-(phenylmethyl)carbazol-4-yl]oxy]acetyl]-L-glutamic acid dimethyl ester

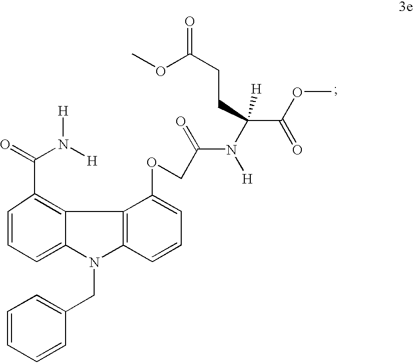

N-[[[5-Carbamoyl-9-(phenylmethyl)carbazol-4-yl]oxy]acetyl]-L-glutamic acid

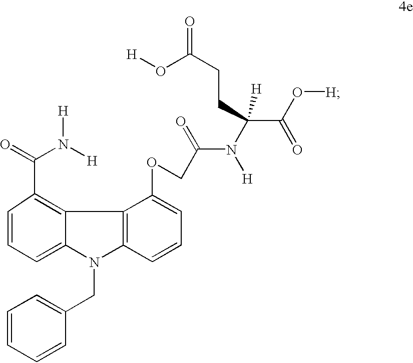

N-[[[5-Carbamoyl-9-(phenylmethyl)carbazol-4-yl]oxy]acetyl]-L-methionine

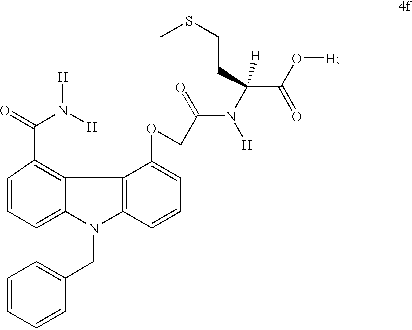

N-[[[5-Carbamoyl-9-(phenylmethyl)carbazol-4-yl]oxy]acetyl]-L-methionine methyl ester

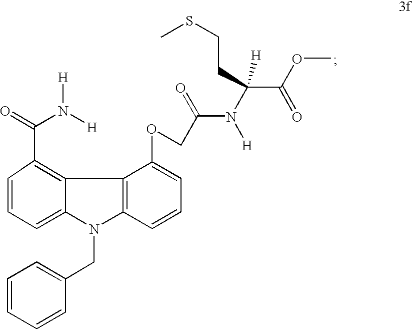

N-[[[5-Carbamoyl-9-(phenylmethyl)carbazol-4-yl]oxy]acetyl]-L-methionine

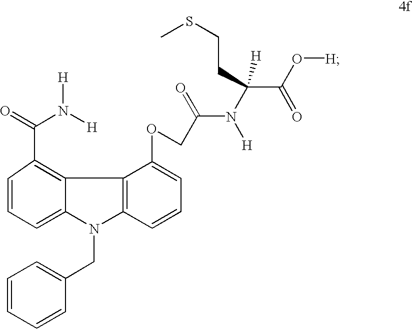

N-[[[5-Carbamoyl-9-(phenylmethyl)carbazol-4-yl]oxy]acetyl]-L-phenylalanine

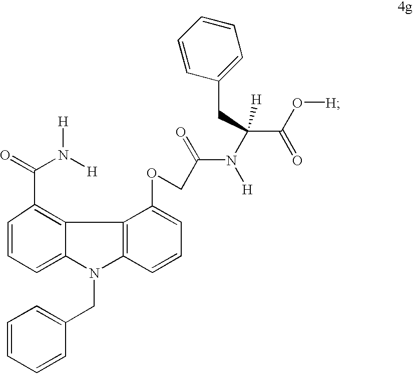

N-[[[5-Carbamoyl-9-(phenylmethyl)carbazol-4-yl]oxy]acetyl]-L-phenylalanine methyl ester

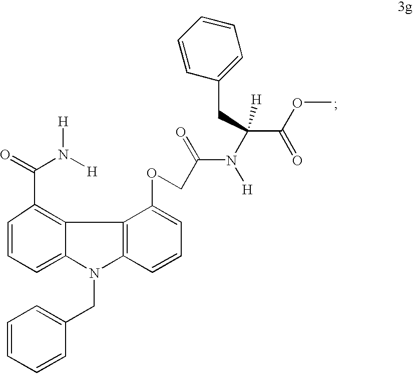

N-[[[5-Carbamoyl-9-(phenylmethyl)carbazol-4-yl]oxy]acetyl]-L-phenylalanine

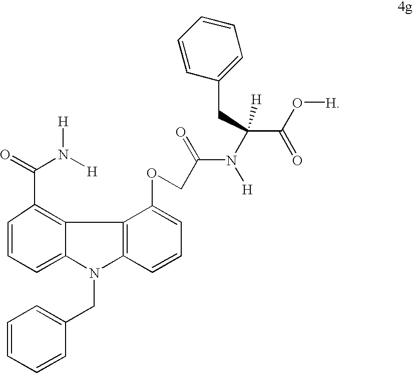

Compounds useful as starting materials to prepare compounds of the present invention have been disclosed along with their methods of preparation, in U.S. patent application Ser. No. 09/063,066 filed Apr. 21, 1998 (titled, “Substituted Carbazoles and 1,2,3,4-Tetrahydrocarbazoles”), the entire disclosure of which is incorporated herein by reference. These compounds useful as starting material to prepare compounds of the present invention include but are not limited to:

5-hydroxy-7-(5-cyanopentyl)-9-methyl-1,2,3,4-tetrahydrocarbazole-4-carboxamide;

6-(2-carboxyethoxy)-8-methoxy-9-cyclopentylmethyl-1,2,3,4-tetrahydrocarbazole-4-carboxamide;

5-(3-phenylpropoxy)-7-ethoxy-9-butyl-1,2,3,4-tetrahydrocarbazole-4-carboxamide;

6-(2-phosphonoethoxy)-8-phenylhexyl-9-(cyclotetradecyl)methyl-1,2,3,4-tetrahydrocarbazole-4-carboxamide;

5-ethoxycarbonylmethoxy-8-(5-carbamoylpent-1-yl)-9-(3,5-dipropylphenyl)methyl-1,2,3,4-tetrahydrocarbazole-4-carboxamide;

6-(diethoxyphosphonyl)methoxy-9-(4-methoxyphenyl)methyl-1,2,3,4-tetrahydrocarbazole-4-carboxamide;

6-(3-(4-carboxyphenyl)prop-1-yl)oxy-8-heptyl-9-(3-phenylethyl)phenyl)methyl-1,2,3,4-tetrahydrocarbazole-4-carboxamide;

6-(2-propoxycarbonyl)ethoxy-8-(3-(N,N-dimethylamino)prop-1-yl)-9-methyl-1,2,3,4-tetrahydrocarbazole-4-carboxamide;

5-(di-t-butoxyphosphonyl)methoxy-7-nonyl-9-(3-propylthiophenyl)methyl-1,2,3,4-tetrahydrocarbazole-4-carboxamide;

5-(2-(3-methoxycarbonyl)phenyl)ethoxy-7-pentyl-9-methyl-1,2,3,4-tetrahydrocarbazole-4-carboxamide;

6-hydroxy-8-(4-(N,N-diethylamino)but-1-yl)-9-(3-fluorophenyl)methyl-1,2,3,4-tetrahydrocarbazole-4-carboxamide;

6-(2-phenylethoxy)-9-(2-phenylphenyl)methyl-1,2,3,4-tetrahydrocarbazole-4-carboxamide;

Particularly preferred compounds useful as starting materials for preparation of compounds for the treatment of inflammation are represented by the formulae (Xe) and (XIe) below:

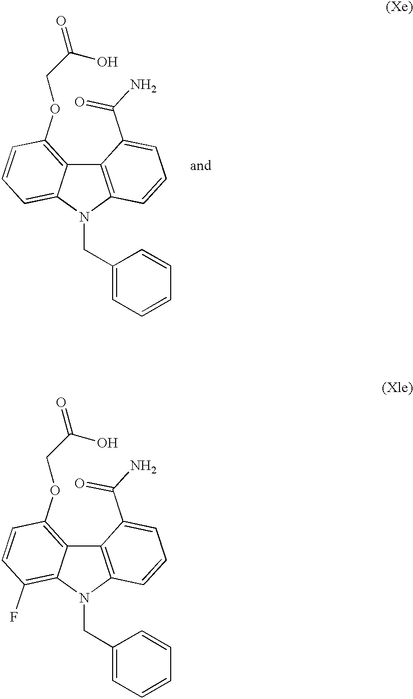

For all of the above compounds of the carbazole or tetrahydrocarbazole type it is advantageous to use them in their (i) acid form where possible, or (ii) pharmaceutically acceptable (e.g., Na, K) form, or (iii) prodrugs derivatives (e.g., methyl ester, ethyl ester, n-butyl ester, morpholino ethyl ester).

Prodrugs are derivatives of sPLA₂ inhibitors used in the method of the invention which have chemically or metabolically cleavable groups and become by solvolysis or under physiological conditions the compounds of the invention which are pharmaceutically active in vivo. Derivatives of the compounds of this invention have activity in both their acid and base derivative forms, but the acid derivative form often offers advantages of solubility, tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine. Simple aliphatic or aromatic esters derived from acidic groups pendent on the compounds of this invention are preferred prodrugs. In some cases it is desirable to prepare double ester type prodrugs such as (acyloxy) alkyl esters or ((alkoxycarbonyl)oxy)alkyl esters. Specific preferred prodrugs are ester prodrugs inclusive of methyl ester, ethyl ester, n-propyl ester, isopropyl ester, n-butyl ester, sec-butyl, tert-butyl ester, N,N-diethylglycolamido ester, and morpholino-N-ethyl ester. Methods of making ester prodrugs are disclosed in U.S. Pat. No. 5,654,326. Additional methods of prodrug synthesis are disclosed in U.S. Provisional Patent Application Serial No. 60/063,280 filed Oct. 27, 1997 (titled, N,N-diethylglycolamido ester Prodrugs of Indole sPLA₂ Inhibitors), the entire disclosure of which is incorporated herein by reference; U.S. Provisional Patent Application Serial No. 60/063,646 filed Oct. 27, 1997 (titled, Morpholino-N-ethyl Ester Prodrugs of Indole sPLA₂ Inhibitors), the entire disclosure of which is incorporated herein by reference; and U.S. Provisional Patent Application Serial No. 60/063,284 filed Oct. 27, 1997 (titled, Isopropyl Ester Prodrugs of Indole sPLA₂ Inhibitors), the entire disclosure of which is incorporated herein by reference.

Carbazole and tetrahydrocarbazole sPLA₂ inhibitor compounds useful for practicing the method of the invention may be made by the following general methods:

Compounds of formula I where R^{2′}, is O(CH₂)ₜR⁵ and R⁵ is an “acylamino acid group are prepared by converting the free acid or derivatives of the compounds of formula XIe, Xe, or other starting material compounds described previously, to the corresponding compounds of the present invention as shown in Scheme 1

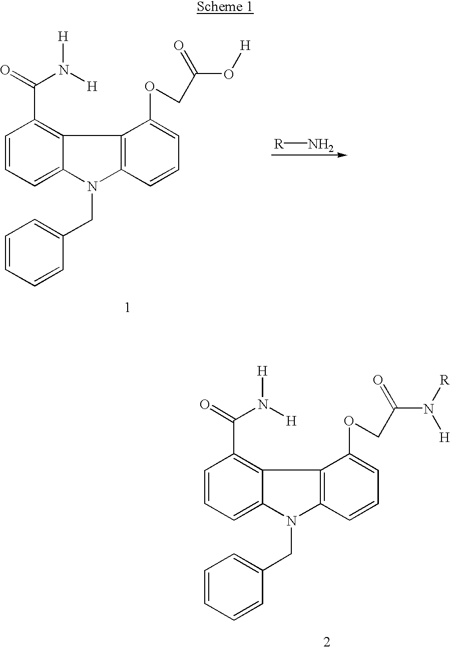

Compounds of the present invention wherein R^{2′} is [tricyclic]-Lₕ-(acylamino acid) where the acylamino acid group is represented by the formula

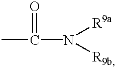

wherein R^{9a} is selected from the group consisting of H, (C₁-C₆)alkyl, (C₁-C₆)alkoxy, heteroaryl and aryl, —CF₃; and wherein NR^{9b} is an amino acid residue of either a natural or unnatural amino acid with the nitrogen atom being part of the amino group of the amino acid, may be prepared by coupling the tetrahydrocarbazole or the carbazole of formula Ie, If, or Ig with an amino acid or protected amino acid such as the methyl ester as shown for example in Scheme 2

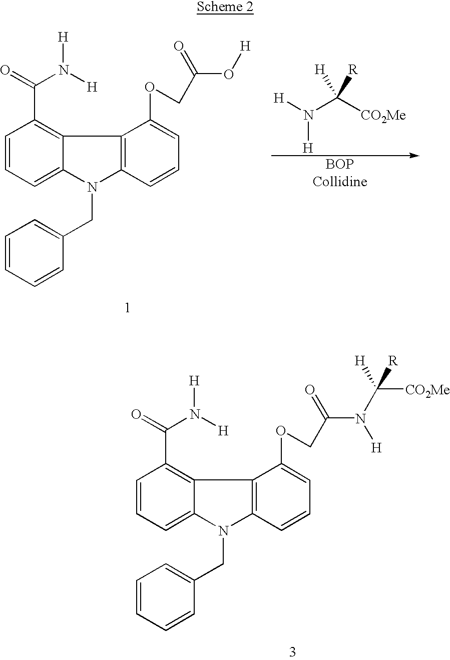

The compounds of the present invention wherein R^{5′} is the group —Lₕ-acylamino acid group are prepared by base catalyzed amino acid coupling reactions. The reaction involves coupling of the free acid or acid derivative as shown in for example, Scheme 2, with a N-terminus amino acid (i.e, the C-terminal has been capped by derivatization to the ester for example). The reaction may be catalyzed by a base. In general a base which minimizes the incidence of racemization, i.e, a hindered base is employed. Particularly preferred for this reaction is a tertiary base such as collidine. Various amino acid coupling agent may may be utilized to afford the coupled product of formula I or derivative thereof. Coupling agents include but are not limited to HOBT (hydroxy benzotriazole), dicyclohexyl carbodiimide (DCCI), Castro's benzotriazolyloxy-tris-(dimethylamino)phosphonium hexafluorophosphate (BOP) or salts of BOP i.e., pyrrolidinium BOP (PyBOP). Most preferred for the present invention is BOP. One of skill in the art I aware that other coupling agents and coupling reactions can be employed. For the present invention utilizing BOP, an equivalent of the BOP reagent is added to a mixture (preferably 1:1) of the starting material and the N-terminus amino acid in a suitable solvent. A suitable solvent is inert to the substrates under the reaction condition. A preferred solvent is DMF.

The coupling reaction may be performed at about 0-150° C., depending on the solvent, and other factors such as reactivity of substrate, etc. The reaction is preferably performed at about room temperature. The reaction time is from about 10 minutes to about 48 hours. A preferred reaction time is from about 1 to 3 hours. The reaction product may be isolated after removing the solvent and following chromatography. Other isolation protocols known to one of skill in the art are not precluded.

The protected amino acid derivative compounds as shown in Scheme 2, may be further hydrolyzed to afford the free acid compounds as shown for example in scheme 3 (below); the compounds and processes of which are further embodiments of the present invention.

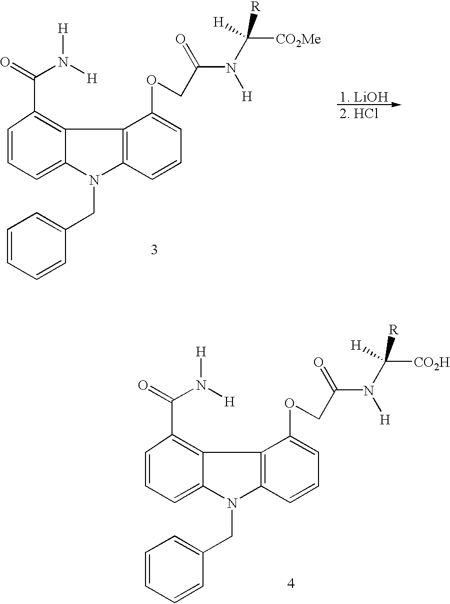

Protecting groups of the R^{5′} substituent, particularly when R^{5′} is —Lₕ-(acylamino acid) may be removed by hydrolysis. Preferred is basic hydrolysis employing aqueous bases for example NaOH, KOH, LiOH, Ca(OH)₂, etc. Particularly preferred is lithium hydroxide. The hydrolysis reaction is performed in an aqueous solvent or a mixture of aqueous solvents. Preferred is a mixture of THF, methanol and water. The reaction is preferably performed at about room temperature or colder. The product may be isolated as the salt or as the free acid after acid hydrolysis (e.g., 5N HCl).

A further embodiment of the present invention and compounds thereof are prepared by subjecting the free amino acid product of step 3 to a subsequent coupling reaction as in Scheme 2 to form the di-peptide, tri-peptide and polypeptide. Alternatively the process of Scheme 2 can be accomplished by utilizing a peptide (mono, di, tri or poly) to form the corresponding compounds of formula I. Procedure for generating the peptides and methods of coupling analogous to the above described are known tom one of skill in the art and are also provided in Amino Acid and Peptide Synthesis, John Jones, Oxford Chemistry Primers, Stephen G. Davis Editor, Oxford University Press Inc. New York, N.Y. (1992).

### Preparation of Starting Material Compounds

The starting material compounds of formula Ie where Z is cyclohexene are prepared according to the following reaction Schemes Ig(a)and (c).

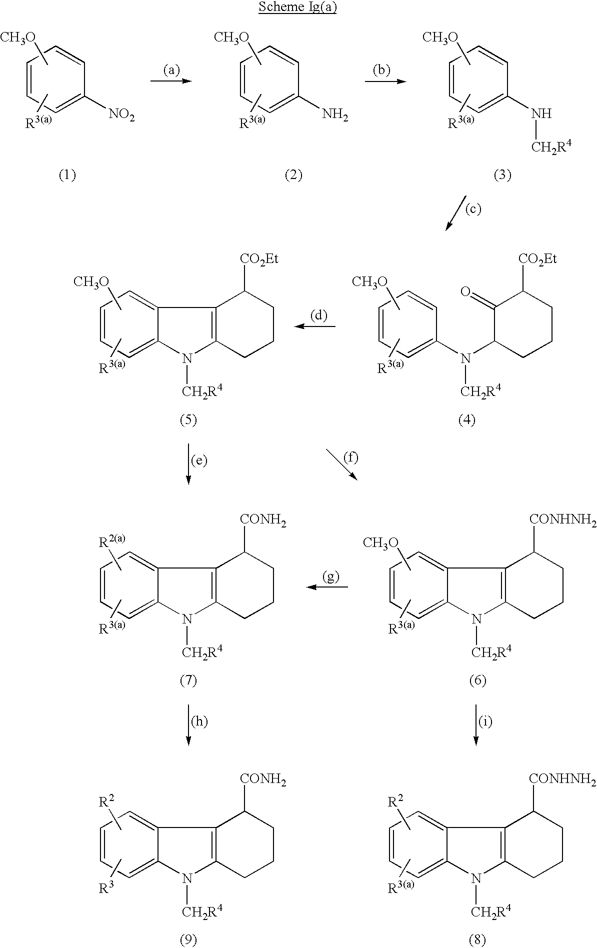

wherein;

R¹ is —NH₂, R³(a) is H, —O(C₁-C₄)alkyl, halo, —(C₁-C₆) alkyl, phenyl, —(C₁-C₄) alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl, —(C₁-C₆)alkoxy(C₁-C₆)alkyl, —(C₁-C₆)alkoxy(C₁-C₆)alkenyl; or —(CH₂)ₙR⁸ where R⁸ is H, —CONH₂, —NR⁹R¹⁰, —CN or phenyl where R⁹ and R¹⁰ are independently hydrogen, —CF₃, phenyl, —(C₁-C₄)alkyl, —(C₁-C₄)alkylphenyl or -phenyl(C₁-C₄)alkyl and n is 1 to 8;

when R¹ is —NHNH₂, R³(a) is H, —O(C₁-C₄)alkyl, halo, (C₁-C₆)alkyl, phenyl, —(C₁-C₄)alkylphenyl; phenyl substituted with —(C₁-C₆)alkyl, halo or —CF₃; —CH₂OSi(C₁-C₆)alkyl, furyl, thiophenyl, —(C₁-C₆)hydroxyalkyl, —(C₁-C₆)alkoxy(C₁-C₆)alkyl, —(C₁-C₆)alkoxy(C₁-C₆)alkenyl; or —(CH₂)ₙR⁸ where R⁸ is H, —NR⁹R¹⁰, —CN or phenyl where R⁹ and R¹⁰ are independently hydrogen, —CF₃, phenyl, —(C₁-C₄)alkyl, —(C₁-C₄)alkylphenyl or -phenyl(C₁-C₄)alkyl and n is 1 to 8;

R² (a) is —OCH₃ or —OH.

An appropriately substituted nitrobenzene (1) can be reduced to the aniline (2) by treatment with a reducing agent, such as hydrogen in the presence of Pd/C, preferably at room temperature.

Compound (2) is N-alkylated at temperatures of from about 0 to 20° C. using an alkylating agent such as an appropriately substituted aldehyde and sodium cyanoborohydride to form (3). Alternately, an appropriately substituted benzyl halide may be used for the first alkylation step. The resulting intermediate is further N-alkylated by treatment with 2-carbethoxy-6-bromocyclohexanone, preferably at temperatures of about 80° C. to yield (4) or by treatment with potassium hexamethyldisilazide and the bromoketoester.

The product (4) is cyclized to the tetrahydrocarbazole (5) by refluxing with ZnCl₂ in benzene for from about 1 to 2 days, preferably at 80°C., (See Julia, M.; Lenzi, J. Preparation d'acides tetrahydro-1,2,3,4-carbazole-1 ou -4.

Bull. Soc. Chim. France, 1962, 2262-2263).

Compound (5) is converted to the hydrazide (6) by treatment with hydrazine at temperatures of about 100° C., or to the amide (7) by reacting with methylchloroaluminum amide in benzene (see Levin, J. I.; Turos, E.; Weinreb, S. M. An alternative procedure for the aluminum-mediated conversion of esters to amides. Syn. Comm., 1982, 12, 989-993). Alternatively, (7) may be produced by treatment of (6) with Raney nickel active catalyst.

It will be readily appreciated that when R³(a) is:

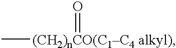

conversion to the amide will also be achieved in this procedure.

Compounds (6) and (7) may be dealkylated, preferably at 0° C. to room temperature, with a dealkylating agent, such as boron tribromide or sodium thioethoxide, to give compound (7) where R²(a) is —OH, which may then be further converted to compound (9), by realkylating with a base, such as sodium hydride, and an alkylating agent, such as Br(CH₂)ₘR⁵, where R⁵ is the carboxylate or phosphonic diester or nitrile as defined above.

Conversion of R² to the carboxylic acid may be accomplished by treatment with an aqueous base. When R² is nitrile, conversion to the tetrazole may be achieved by reacting with tri-butyl tin azide or conversion to the carboxamide may be achieved by reacting with basic hydrogen peroxide. When R² is the phosphonic diester, conversion to the acid may be achieved by reacting with a dealkylating agent such as trimethylsilyl bromide. The monoester may be accomplished by reacting the diester with an aqueous base.

When R² and R³ are both methoxy, selective demethylation can be achieved by treating with sodium ethanethiolate in dimethylformamide at 100° C.

An alternative synthesis of intermediate (5) is shown in Scheme I(b), as follows.

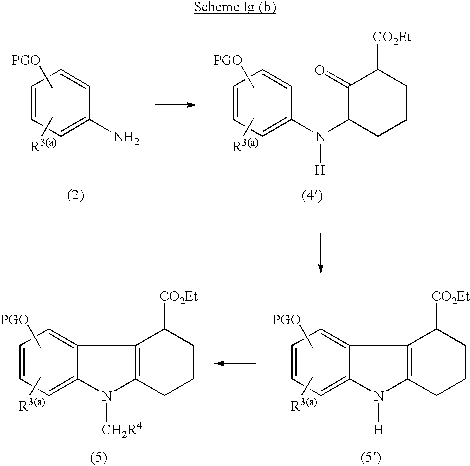

where PG is a protecting group;

R^{3a} is as defined in Scheme 1, above.

The aniline (2) is N-alkylated with 2-carbethoxy-6-bromocyclohexanone in dimethyl formamide in the presence of sodium bicarbonate for 8-24 hours at 50° C. Preferred protecting groups include methyl, carbonate, and silyl groups, such as t-butyldimethylsilyl. The reaction product (4′) is cyclized to (5′) using the ZnCl₂ in benzene conditions described in Scheme I(a), above. N-alkylation of (5′) to yield (5) is accomplished by treatment with sodium hydride and the appropriate alkyl halide in dimethylformamide at room temperature for 4-8 hours.

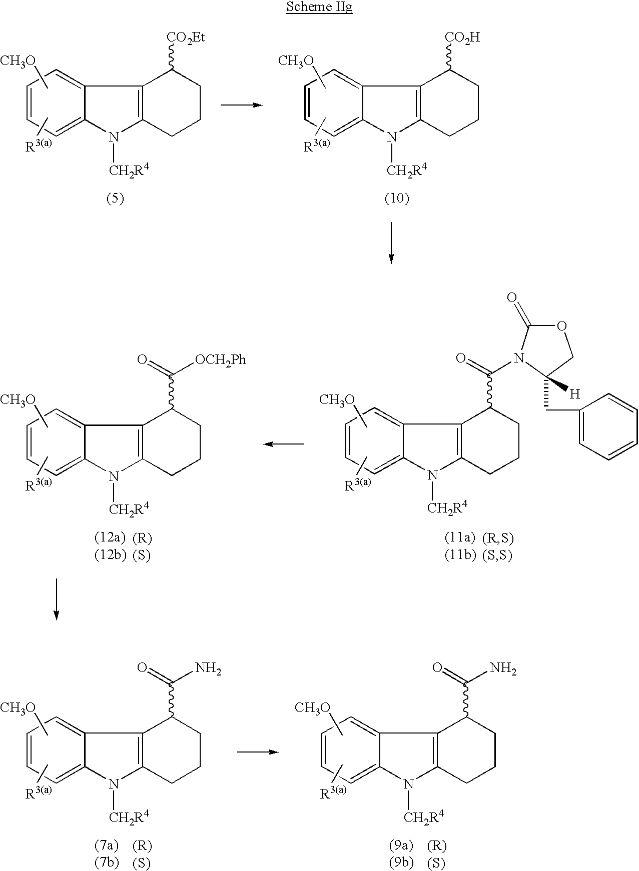

R³(a) is as defined in Scheme Ig.

As discussed in Scheme I above, carbazole (5) is hydrolyzed to the carboxylic acid (10) by treatment with an aqueous base, preferably at room temperature to about 100° C. The intermediate is then converted to an acid chloride utilizing, for example, oxalyl chloride and dimethylformamide, and then further reacted with a lithium salt of (S) or (R)-4-alkyl-2-oxazolidine at a temperature of about −75° C., to give (11a) and (11b), which are separable by chromatography.

The diastereomers are converted to the corresponding enantiomeric benzyl esters (12) by brief treatment at temperatures of about 0° C. to room temperature with lithium benzyl oxide. See Evans, D. A.; Ennis, M. D.; Mathre, D. J. Asymmetric alkylation reactions of chiral imide enolates. A practical approach to the enantioselective synthesis of alpha-substituted carboxylic acid derivatives. J. Am. Chem. Soc., 1982, 104, 1737-1738. The esters (12) are then converted to (7) preferably by treatment with methylchloroaluminum amide (see Levin, J. I.; Turos, E.; Weinreb, S. M. An alternative procedure for the aluminum-mediated conversion of esters to amides is disclosed in Syn. Comm., 1982, 12, 989-993. Alternately, the conversion of esters to amides may be accomplished by hydrogenation using, for example, hydrogen and palladium on carbon, as described above, to make the acid and then reacting with an acyl azide, such as diphenylphosphoryl azide followed by treatment with ammonia. Using the procedure described above in Scheme I, preparation of compound (9a) or (9b) may be accomplished.

Compounds of formula Ie where Z is phenyl can be prepared as follows in Schemes III(a)-(f), below.

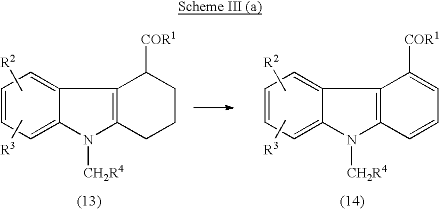

A 1,2,3,4-tetrahydrocarbazole-4-carboxamide or 4-carboxhydrazide (13) is dehydrogenated by refluxing in a solvent such as carbitol in the presence of Pd/C to produce the carbazole-4-carboxamide. Alternately, treatment of (13) with DDQ in an appropriate solvent such as dioxane yields carbazole (14).

Depending on the substitution pattern, oxidation as described above may result in de-alkylation of the nitrogen. For example when R³ is substituted at the 8-position with methyl, oxidation results in dealkylation of the nitrogen which may be realkylated by treatment with sodium hydride and the appropriate alkyl halide as described in Scheme I(a) above to prepare the desired product (14).

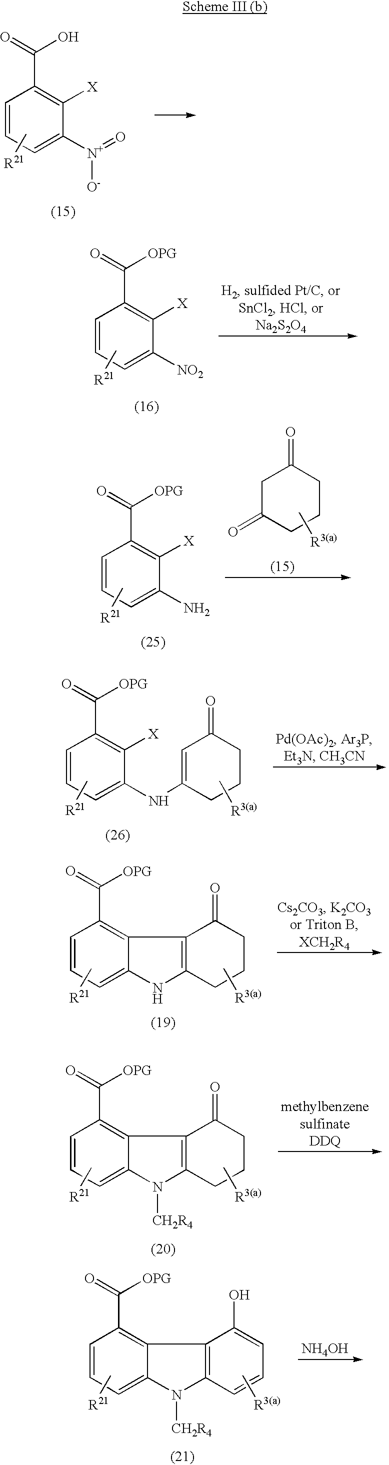

Benzoic acid derivative (16) where X is preferably chlorine, bromine or iodine and the protecting group is preferably —CH₃, are reduced to the corresponding aniline (25) with a reducing agent, such as stannous chloride in the presence of acid under the general conditions of Sakamoto et al, Chem Pharm. Bull. 35 (5), 1823-1828 (1987).

Alternatively, reduction with sodium dithionite in the presence of a base, such as sodium carbonate in a non-interfering solvent, such as water, ethanol, and/or tetrahydrofuran affords the starting material compound (16).

Alternatively, reduction by hydrogenation over a sulfide platinum catalyst supported on carbon with hydrogen at 1 to 60 atmospheres in a non-interfering solvent, preferably ethyl acetate, to form the starting material compound (16).

The reactions are conducted at temperatures from about 0 to 100° C. preferably at ambient temperature, and are substantially complete in about 1 to 48 hours depending on conditions.

The aniline (25) and dione (15) are condensed under dehydrating conditions, for example, using the general procedure of Iida, et al., (J of Org. Chem. 45, 2938 (1980)), with or without a non-interfering solvent, such as toluene, benzene, or methylene chloride, under dehydrating conditions at a temperature about 10 to 150° C. The water formed in the process can be removed by distillation, azeotropic removal via a Dean-Stark apparatus, or the addition of a drying agent, such as molecular sieves, magnesium sulfate, calcium carbonate, sodium sulfate, and the like.

The process can be performed with or without a catalytic amount of an acid, such a p-toluenesulfonic acid or methanesulfonic acid. Other examples of suitable catalysts include hydrochloric acid, phenylsulfonic acid, calcium chloride, and acetic acid.

Examples of other suitable solvents include tetrahydrofuran, ethyl acetate, methanol, ethanol, 1,1,2,2-tetrachloroethane, chlorobenzene, bromobenzene, xylenes, and carbon tetrachloride.

The condensation of the instant process is preferably carried out neat, at a temperature about 100 to 150° C. with the resultant water removed by distillation via a stream of inert gas, such as, nitrogen or argon.

The reaction is substantially complete in about 30 minutes to 24 hours.

Intermediate (26) may then be readily cyclized in the presence of a palladium catalyst, such as Pd(OAc)₂ or Pd(PPh₃)₄ and the like, a phosphine, preferably a trialkyl- or triarylphosphine, such as triphenylphosphine, tri-o-tolylphosphine, or tricyclohexylphosphine, and the like, a base, such as, sodium bicarbonate, triethylamine, or diisopropylethylamine, in a non-interfering solvent, such as, acetonitrile, triethylamine, or toluene at a temperature about 25 to 200° C. to form (19).

Examples of other suitable solvents include tetrahydrofuran, benzene, dimethylsulfoxide, or dimethylformamide.

Examples of other suitable palladium catalysts include Pd(PPh₃)Cl₂, Pd(OCOCF₃)₂, [(CH₃C₆H₄)₃P]₂PdCl₂, [(CH₃CH₂)₃P]₂PdCl₂, [(C₆H₁₁)₃P]₂PdCl₂, and [(C₆H₅)₃P]₂PdBr₂.

Examples of other suitable phosphines include triisopropylphosphine, triethylphosphine, tricyclopentylphosphine, 1,2-bis(diphenylphosphino)ethane, 1,3-bis(diphenylphosphino)propane, and 1,4-bis(diphenylphosphino)butane.

Examples of other suitable bases include triisopropyl amine, 2,2,6,6-tetramethylpiperidine, 1,5-diazabicyclo[2.2.2]octane (DABCO), 1,8-diazabicyclo[5.4.0]undec-7-ene (DBU), 1,5-diazabicyclo[4.3.0]non-5-ene, (DBN) sodium carbonate, potassium carbonate, and potassium bicarbonate.

The cyclization of the instant process is preferably carried out with palladium(II)acetate as catalyst in the presence of either triphenylphosphine, tri-o-tolylphosphine, 1,3-bis(diphenylphosphino)propane, or tricyclohexylphosphine in acetonitrile as solvent and triethylamine as base at a temperature about 50 to 150° C. The reaction is substantially complete in about 1 hour to 14 days.

Alternatively, a preferred process for cyclization consists of the reaction of intermediate (26) with a palladacycle catalyst such as trans-di(μ-acetato)-bis[o-(di-o-tolylphosphino)benzyl]dipalladium (II) in a solvent such as dimethylacetamide (DMAC) at 120-140° C. in the presence of a base such as sodium acetate.

Intermediate (19) may be alkylated with an alkylating agent XCH₂R₄, where X is halo in the presence of a base to form (20). Suitable bases include potassium carbonate, sodium carbonate, lithium carbonate, cesium carbonate, sodium bicarbonate, potassium bicarbonate, potassium hydroxide, sodium hydroxide, sodium hydride, potassium hydride, lithium hydride, and Triton B (N-benzyltrimethylammonium hydroxide).

The reaction may or may not be carried out in the presence of crown ether. Potassium carbonate and Triton B are preferred. The amount of alkylating agent is not critical, however, the reaction is best accomplished using an excess of alkyl halide relative to the starting material.

A catalytic amount of an iodide, such as sodium iodide or lithium iodide may or may not be added to the reaction mixture. The reaction is preferably carried out in an organic solvent, such as, acetone, dimethylformamide, dimethylsulfoxide, or acetonitrile. Other suitable solvents include tetrahydrofuran, methyl ethyl ketone, and t-butyl methyl ether.

The reaction is conducted at temperatures from about −10 to 100° C. preferably at ambient temperature, and is substantially complete in about 1 to 48 hours depending on conditions. Optionally, a phase transfer reagent such as tetrabutylammonium bromide or tetrabutylammonium chloride may be employed.

Intermediate (20) may by dehydrogenated by oxidation with 2,3-dichloro-5,6-dicyano-1,4-benzoquinone in a non-interfering solvent to form (21).

Suitable solvents include methylene chloride, chloroform, carbon tetrachloride, diethyl ether, methyl ethyl ketone, and t-butyl methyl ether. Toluene, benzene, dioxane, and tetrahydrofuran are preferred solvents. The reaction is carried out at a temperature about 0 to 120° C. Temperatures from 50 to 120° C. are preferred. The reaction is substantially complete in about 1 to 48 hours depending on conditions.

Intermediate (21) may be aminated with ammonia in the presence of a non-interfering solvent to form compound (22). Ammonia may be in the form of ammonia gas or an ammonium salt, such as ammonium hydroxide, ammonium acetate, ammonium trifluoroacetate, ammonium chloride, and the like. Suitable solvents include ethanol, methanol, propanol, butanol, tetrahydrofuran, dioxane, and water. A mixture of concentrated aqueous ammonium hydroxide and tetrahydrofuran or methanol is preferred for the instant process. The reaction is carried out at a temperature about 20 to 100° C. Temperatures from 50 to 60° C. are preferred. The reaction is substantially complete in about 1 to 48 hours depending on conditions.

Alkylation of (22) is achieved by treatment with an alkylating agent of the formula XCH₂R⁹ where X is halo and R⁷⁰ is —CO₂R⁷¹, —SO₃R⁷¹, —P(O)(OR⁷¹)₂, or —P(O)(OR⁷¹)H, where R⁷¹ is an acid protecting group or a prodrug function, in the presence of a base in a non-interfering solvent to form (23). Methyl bromoacetate and t-butyl bromoacetate are the preferred alkylating agents.

Suitable bases include potassium carbonate, sodium carbonate, lithium carbonate, cesium carbonate, sodium bicarbonate, potassium bicarbonate, potassium hydroxide, sodium hydroxide, sodium hydride, potassium hydride, lithium hydride, and Triton B (N-benzyltrimethylammonium hydroxide). The reaction may or may not be carried out in the presence of a crown-ether. Cesium carbonate and Triton B are preferred bases.

The amount of alkylating agent is not critical, however, the reaction is best accomplished using an excess of alkyl halide relative to the starting material. The reaction is preferably carried out in an organic solvent, such as, acetone, dimethylformamide, dimethylsulfoxide, or acetonitrile. Other suitable solvents include tetrahydrofuran, methyl ethyl ketone, and t-butyl methyl ether.

The reaction is conducted at temperatures from about −10 to 100° C. preferably at ambient temperature, and is substantially complete in about 1 to 48 hours depending on conditions. Optionally, a phase transfer reagent such as tetrabutylammonium bromide or tetrabutylammonium chloride may be employed.

Intermediate (23) may be optionally hydrolyzed with a base or acid to form desired product (24) and optionally salified.

Hydrolysis of (23) is achieved using a base such as sodium hydroxide, potassium hydroxide, lithium hydroxide, aqueous potassium carbonate, aqueous sodium carbonate, aqueous lithium carbonate, aqueous potassium bicarbonate, aqueous sodium bicarbonate, aqueous lithium bicarbonate, preferably sodium hydroxide and a lower alcohol solvent, such as, methanol, ethanol, isopropanol, and the like. Other suitable solvents include acetone, tetrahydrofuran, and dioxane.

Alternatively, the acid protecting group may be removed by organic and inorganic acids, such as trifluoroacetic acid and hydrochloric acid with or without a non-interfering solvent. Suitable solvents include methylene chloride, tetrahydrofuran, dioxane, and acetone. The t-butyl esters are preferably removed by neat trifluoroacetic acid.

The reaction is conducted at temperatures from about −10 to 100° C. preferably at ambient temperature, and is substantially complete in about 1 to 48 hours depending on conditions.

The starting material (16) is prepared by esterifying compound (15) with a alkyl halide=XPG; where X is halo and PG is an acid protecting group, in the presence of a base, preferably potassium carbonate or sodium carbonate, in a non-interfering solvent, preferably dimethylformamide or dimethylsulfoxide. The preferred alkyl halide is methyl iodide. The reaction is conducted at temperatures from about 0 to 100° C. preferably at ambient temperature, and is substantially complete in about 1 to 48 hours depending on conditions.

Alternatively the starting material (16) may be prepared by condensation with an alcohol HOPG, where PG is an acid protecting group, in the presence of a dehydrating catalyst such as, dicyclohexylcarbodiimide (DCC) or carbonyl diimidazole.

In addition, U.S. Pat. No. 4,885,338 and Jpn. Kokai Tokkyo Koho 05286912, Nov 1993 Hesei teach a method for preparing 2-fluoro-5-methoxyaniline derivatives useful for the preparation of a starting material for the purpose of the present invention.

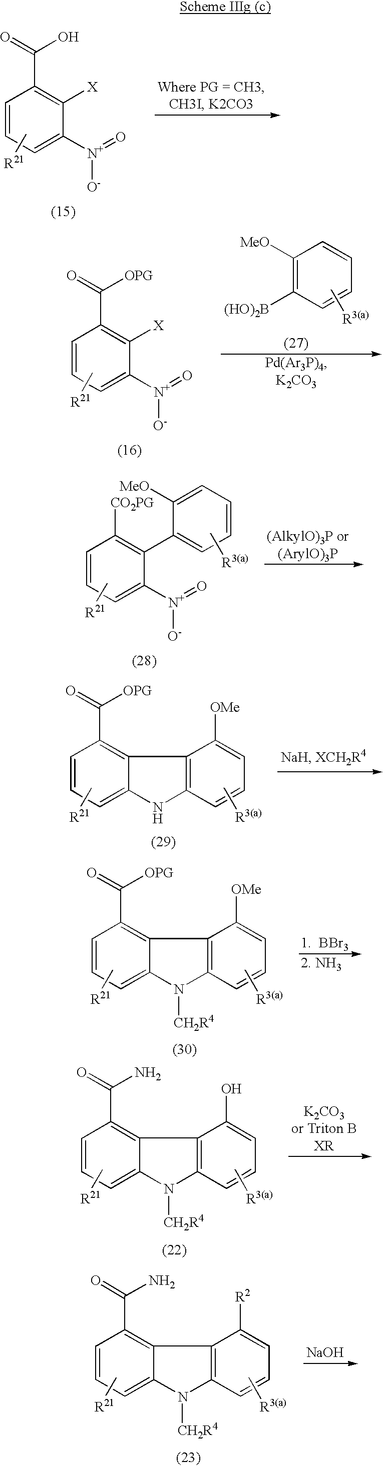

R is as defined in Scheme IIIg(b),

R³(a) is as defined in Scheme Ig(a), above; and X is halo.

Benzoic acid derivatives (16) (X═Cl, Br, or I) and boronic acid derivative (27) (either commercially available or readily prepared by known techniques from commercially available starting materials) are condensed under the general procedure of Miyaura, et al., N. Miyaura, et al., Synth. Commun. 11, 513 (1981)or Trecourt, et al., F. Trecourt, et al., Tetrahedron, 51, 11743 6) in the presence of a palladium catalyst, such as Pd(Ph₃P)₄, a base, such as sodium bicarbonate, in an inert solvent, such as THF, toluene or ethanol, to afford compound (28).

Compound (28) is converted to the carbazole product (29) by treatment with a trialkyl or triaryl phosphite or phosphine, such as, triethylphosphite or triphenyl phosphine, according to the general procedure of Cadogan, et al. (see J. Cadogan et al., J. Chem. Soc., 4831 (1965)

Compound (29) is N-alkylated with an appropriately substituted alkyl or aryl halide XCH₂R⁴ in the presence of a base, such as sodium hydride or potassium carbonate, in a non-interfering solvent, such as toluene, dimethylformamide, or dimethylsulfoxide to afford carbazole (30).

Compound (30) is converted to the corresponding amide (22) by treatment with boron tribromide or sodium thioethoxide, followed by ammonia or an ammonium salt, such as ammonium acetate, in an inert solvent, such as water or alcohol, or with methylchloroaluminum amide in an inert solvent, such as toluene, at a temperature between 0 to 110° C.

When R³(a) is substituted at the 8-position with chloro, de-alkylation of (30) with boron tribromide results in de-benzylation of the nitrogen as described above. Alkylation may be readily accomplished in a two step process. First, an O-alkylation by treatment with a haloalkyl acetate such as methyl bromo acetate using sodium hydride in tetrahydrofuran, followed by N-alkylation using for example a base such as sodium hydride and an appropriately substituted alkyl or aryl halide in dimethoxy formamide. Compound (22) can be converted to product carbazole product (24) as described previously in Scheme IIIg(b) above.

Conversion to the desired prodrug may be accomplished by techniques known to the skilled artisan, such as for example, by treatment with a primary or secondary halide to make an ester prodrug.

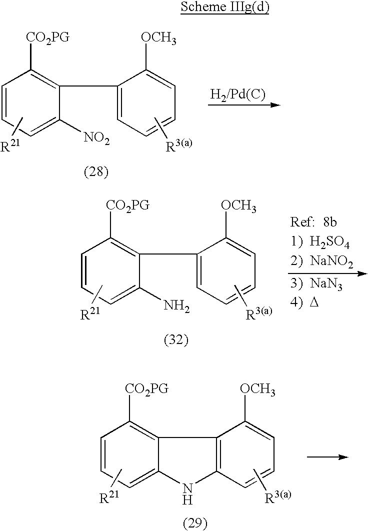

Alternatively, reduction of the nitro group of compound (28) with a reducing agent, such as hydrogen in the presence of palladium on carbon, in a non-interfering solvent, such as ethanol, at 1 to 60 atmospheres, at a temperature of 0 to 60° C. affords the corresponding aniline (32). Compound (32) is converted to the carbazole (29) according to the general procedure described by Trecourt, et al. (Ref 8b). The aniline is treated with sulfuric acid and sodium nitrite, followed by sodium azide to form an intermediate azide which is cyclized to carbazole (29) by heating in an inert solvent, such as toluene. Compound (29) is converted to carbazole product (24) as described previously in Schemes IIIg(b) and IIIg(c).

Typical starting materials which may be converted to the corresponding compounds of formula I useful in the present invention, by the methods described supra, also include:

5-hydroxy-7-(5-cyanopentyl)-9-methyl-1,2,3,4-tetrahydrocarbazole-4-carboxamide;

6-(2-carboxyethoxy)-8-methoxy-9-cyclopentylmethyl-1,2,3,4-tetrahydrocarbazole-4-carboxamide;

5-(3-phenylpropoxy)-7-ethoxy-9-butyl-1,2,3,4-tetrahydrocarbazole-4-carboxamide; or a pharmaceutically acceptable salt, racemate, solvate, tautomer, optical isomer or prodrug derivative thereof, and others disclosed in U.S. patent application Ser. No. 09/063,066 filed Apr. 21, 1998 (titled, “Substituted Carbazoles and 1,2,3,4-Tetrahydrocarbazoles”), the entire disclosure of which is incorporated herein by reference.

### Therapeutic Use of Tricyclic Compounds

The compounds described herein are believed to achieve their beneficial therapeutic action principally by direct inhibition of human sPLA₂, and not by acting as antagonists for arachidonic acid, nor other active agents below arachidonic acid in the arachidonic acid cascade, such as 5-lipoxygenases, cyclooxygenases, etc.

The method of the invention for inhibiting sPLA₂ mediated release of fatty acids comprises contacting sPLA₂ with a therapeutically effective amount of the compound of Formula (I) or its salt.

The compounds of the invention may be used in a method of treating a mammal (e.g., a human) to alleviate the pathological effects of septic shock, adult respiratory distress syndrome, pancreatitus, trauma, bronchial asthma, allergic rhinitis, and rheumatoid arthritis; wherein the method comprises administering to the mammal a compound of formula (I) in a therapeutically effective amount. A “therapeutically effective” amount is an amount sufficient to inhibit sPLA₂ mediated release of fatty acid and to thereby inhibit or prevent the arachidonic acid cascade and its deleterious products. The therapeutic amount of compound of the invention needed to inhibit sPLA₂ may be readily determined by taking a sample of body fluid and assaying it for sPLA₂ content by conventional methods.

Throughout this document, the person or animal to be treated will be described as a “mammal”, and it will be understood that the most preferred subject is a human. Use of the present compounds in non-human animals is also a contemplated aspect of the invention. It will be understood that the dosage ranges for other animals will necessarily be quite different from the doses administered to humans, and accordingly that the dosage ranges described will be recalculated. For example, a small dog may be only 1/10^{th} of a typical human's size, and it will therefore be necessary for a much smaller dose to be used. The determination of an effective amount for a certain non-human animal is carried out in the same manner described below in the case of humans, and veterinarians are well accustomed to such determinations.

### Pharmaceutical Formulations of the Invention

As previously noted the compounds of this invention are useful for inhibiting sPLA₂ mediated release of fatty acids such as arachidonic acid. By the term, “inhibiting” is meant the prevention or therapeutically significant reduction in release of sPLA₂ initiated fatty acids by the compounds of the invention. By “pharmaceutically acceptable” it is meant the carrier, diluent or excipient must be compatible with the other ingredients of the formulation and not deleterious to the recipient thereof.

In general, the compounds of the invention are most desirably administered at a dose that will generally afford effective results without causing any serious side effects and can be administered either as a single unit dose, or if desired, the dosage may be divided into convenient subunits administered at suitable times throughout the day.

The specific dose of a compound administered according to this invention to obtain therapeutic or prophylactic effects will, of course, be determined by the particular circumstances surrounding the case, including, for example, the route of administration, the age, weight and response of the individual patient, the condition being treated and the severity of the patient's symptoms. Typical daily doses will contain a non-toxic dosage level of from about 0.01 mg/kg to about 50 mg/kg of body weight of an active compound of this invention.

Preferably the pharmaceutical formulation is in unit dosage form. The unit dosage form can be a capsule or tablet itself, or the appropriate number of any of these. The quantity of active ingredient in a unit dose of composition may be varied or adjusted from about 0.1 to about 1000 milligrams or more according to the particular treatment involved. It may be appreciated that it may be necessary to make routine variations to the dosage depending on the age and condition of the patient. The dosage will also depend on the route of administration.

A “chronic” condition means a deteriorating condition of slow progress and long continuance. As such, it is treated when it is diagnosed and continued throughout the course of the disease. An “acute” condition is an exacerbation of short course followed by a period of remission. In an acute event, compound is administered at the onset of symptoms and discontinued when the symptoms disappear.

Pancreatitis, trauma-induced shock, bronchial asthma, allergic rhinitis and rheumatoid arthritis may occur as an acute event or a chronic event. Thus, the treatment of these conditions contemplates both acute and chronic forms. Septic shock and adult respiratory distress, on the other hand, are acute conditions treated when diagnosed.

The compound can be administered by a variety of routes including oral, aerosol, rectal, transdermal, subcutaneous, intravenous, intramuscular, and intranasal.

Pharmaceutical formulations of the invention are prepared by combining (e.g., mixing) a therapeutically effective amount of the compounds of the invention together with a pharmaceutically acceptable carrier or diluent therefor. The present pharmaceutical formulations are prepared by known procedures using well known and readily available ingredients.

In making the compositions of the present invention, the active ingredient will usually be admixed with a carrier, or diluted by a carrier, or enclosed within a carrier which may be in the form of a capsule, sachet, paper or other container. When the carrier serves as a diluent, it may be a solid, semi-solid or liquid material which acts as a vehicle, or can be in the form of tablets, pills, powders, lozenges, elixirs, suspensions, emulsions, solutions, syrups, aerosols (as a solid or in a liquid medium), or ointment, containing, for example, up to 10% by weight of the active compound. The compounds of the present invention are preferably formulated prior to administration.

For the pharmaceutical formulations any suitable carrier known in the art can be used. In such a formulation, the carrier may be a solid, liquid, or mixture of a solid and a liquid. Solid form formulations include powders, tablets and capsules. A solid carrier can be one or more substances which may also act as flavoring agents, lubricants, solubilisers, suspending agents, binders, tablet disintegrating agents and encapsulating material.

Tablets for oral administration may contain suitable excipients such as calcium carbonate, sodium carbonate, lactose, calcium phosphate, together with disintegrating agents, such as maize, starch, or alginic acid, and/or binding agents, for example, gelatin or acacia, and lubricating agents such as magnesium stearate, stearic acid, or talc.

In powders the carrier is a finely divided solid which is in admixture with the finely divided active ingredient. In tablets the active ingredient is mixed with a carrier having the necessary binding properties in suitable proportions and compacted in the shape and size desired. The powders and tablets preferably contain from about 1 to about 99 weight percent of the active ingredient which is the novel compound of this invention. Suitable solid carriers are magnesium carbonate, magnesium stearate, talc, sugar lactose, pectin, dextrin, starch, gelatin, tragacanth, methyl cellulose, sodium carboxymethyl cellulose, low melting waxes, and cocoa butter.

Sterile liquid form formulations include suspensions, emulsions, syrups and elixirs.

The active ingredient can be dissolved or suspended in a pharmaceutically acceptable carrier, such as sterile water, sterile organic solvent or a mixture of both. The active ingredient can often be dissolved in a suitable organic solvent, for instance aqueous propylene glycol. Other compositions can be made by dispersing the finely divided active ingredient in aqueous starch or sodium carboxymethyl cellulose solution or in a suitable oil.

The following pharmaceutical formulations 1 through 8 are illustrative only and are not intended to limit the scope of the invention in any way. “Active ingredient”, refers to a compound according to Formula (I), (II) or (III) or a pharmaceutically acceptable salt, solvate, or prodrug thereof.

### Formulation 1

Hard gelatin capsules are prepared using the following ingredients:

| | Quantity | |
|---|---|---|
| | (mg/capsule) | |
| Active ingredient | 250 | |
| Starch, dried | 200 | |
| Magnesium stearate | 10 | |
| Total | 460 | mg |

### Formulation 2

A tablet is prepared using the ingredients below:

| | Quantity | |
|---|---|---|
| | (mg/tablet) | |
| Active ingredient | 250 | |
| Cellulose, microcrystalline | 400 | |
| Silicon dioxide, fumed | 10 | |
| Stearic acid | 5 | |
| Total | 665 | mg |

The components are blended and compressed to form tablets each weighing 665 mg

### Formulation 3

An aerosol solution is prepared containing the following components:

| | Weight |
|---|---|
| Active ingredient | 0.25 |
| Ethanol | 25.75 |
| Propellant 22 (Chlorodifluoromethane) | 74.00 |
| Total | 100.00 |

The active ingredient is mixed with ethanol and the mixture added to a portion of the propellant 22, cooled to −30° C. and transferred to a filling device. The required amount is then fed to a stainless steel container and diluted with the remainder of the propellant. The valve units are then fitted to the container.

### Formulation 4

Tablets, each containing 60 mg of active ingredient, are made as follows:

| | | |
|---|---|---|
| Active ingredient | 60 | mg |
| Starch | 45 | mg |
| Microcrystalline cellulose | 35 | mg |
| Polyvinylpyrrolidone (as 10% solution in | 4 | mg |
| water) | | |
| Sodium carboxymethyl starch | 4.5 | mg |
| Magnesium stearate | 0.5 | mg |
| Talc | 1 | mg |
| Total | 150 | mg |

The active ingredient, starch and cellulose are passed through a No. 45 mesh U.S. sieve and mixed thoroughly. The aqueous solution containing polyvinylpyrrolidone is mixed with the resultant powder, and the mixture then is passed through a No. 14 mesh U.S. sieve. The granules so produced are dried at 50° C. and passed through a No. 18 mesh U.S. sieve. The sodium carboxymethyl starch, magnesium stearate and talc, previously passed through a No. 60 mesh U.S. sieve, are then added to the granules which, after mixing, are compressed on a tablet machine to yield tablets each weighing 150 mg.

### Formulation 5

Capsules, each containing 80 mg of active ingredient, are made as follows:

| | | |
|---|---|---|
| Active ingredient | 80 | mg |
| Starch | 59 | mg |
| Microcrystalline cellulose | 59 | mg |
| Magnesium stearate | 2 | mg |
| Total | 200 | mg |

The active ingredient, cellulose, starch, and magnesium stearate are blended, passed through a No. 45 mesh U.S. sieve, and filled into hard gelatin capsules in 200 mg quantities.

### Formulation 6

Suppositories, each containing 225 mg of active ingredient, are made as follows:

| | | |
|---|---|---|
| Active ingredient | 225 | mg |
| Saturated fatty acid glycerides | 2,000 | mg |
| Total | 2,225 | mg |

The active ingredient is passed through a No. 60 mesh U.S. sieve and suspended in the saturated fatty acid glycerides previously melted using the minimum heat necessary. The mixture is then poured into a suppository mold of nominal 2 g capacity and allowed to cool.

### Formulation 7

Suspensions, each containing 50 mg of active ingredient per 5 ml dose, are made as follows:

| Active ingredient | 50 | mg |
|---|---|---|
| Sodium carboxymethyl cellulose | 50 | mg |
| Syrup | 1.25 | ml |
| Benzoic acid solution | 0.10 | ml |
| Flavor | q.v. | |
| Color | q.v. | |
| Purified water to total | 5 | ml |

The active ingredient is passed through a No. 45 mesh U.S. sieve and mixed with the sodium carboxymethyl cellulose and syrup to form a smooth paste. The benzoic acid solution, flavor and color are diluted with a portion of the water and added, with stirring. Sufficient water is then added to produce the required volume.

### Formulation 8

An intravenous formulation may be prepared as follows:

| | | |
|---|---|---|
| Active ingredient | 100 | mg |
| Isotonic saline | 1,000 | ml |

The solution of the above ingredients generally is administered intravenously to a subject at a rate of 1 ml per minute.

### Assay Experiments

### ASSAY EXAMPLE 1

The following chromogenic assay procedure was used to identify and evaluate inhibitors of recombinant human secreted phospholipase A₂. The assay described herein has been adapted for high volume screening using 96 well microtiter plates. A general description of this assay method is found in the article, “Analysis of Human Synovial Fluid Phospholipase A₂ on Short Chain Phosphatidylcholine-Mixed Micelles: Development of a Spectrophotometric Assay Suitable for a Microtiterplate Reader”, by Laure J. Reynolds, Lori L. Hughes, and Edward A Dennis, Analytical Biochemistry, 204, pp. 190-197, 1992 (the disclosure of which is incorporated herein by reference):

Reagents:

Reaction Buffer—

| | | |
|---|---|---|
| CaCl₂.2H₂O | (1.47 | g/L) |
| KC1 | (7.455 | g/L) |

Bovine Serum Albumin (fatty acid free) (1 g/L)

(Sigma A-7030, product of Sigma Chemical Co. St. Louis Mo., USA)

TRIS HCl (3.94 g/L)

pH 7.5 (adjust with NaOH)

Enzyme Buffer—

0.05 NaOAc.3H₂O, pH 4.5

0.2 NaCl

Adjust pH to 4.5 with acetic acid

DTNB—

5,5′-dithiobis-2-nitrobenzoic acid

Racemic Diheptanoyl Thio—PC

racemic 1,2-bis(heptanoylthio)-1,2-dideoxy-sn-glycero-3-phosphorylcholine

TRITON X-100™ prepare at 6.249 mg/ml in reaction buffer to equal 10 uM

TRITON X-100™ is a polyoxy ethylene non-ionic

detergent supplied by

Pierce Chemical Company,

3747 N. Meridian Road, Rockford, Ill. 61101.

Reaction Mixture—

A measured volume of racemic dipheptanoyl thio PC supplied in chloroform at a concentration of 100 mg/ml is taken to dryness and redissolved in 10 millimolar TRITON X-100™ nonionic detergent aqueous solution. Reaction Buffer is added to the solution, then DTNB to give the Reaction Mixture.

The reaction mixture thus obtained contains 1 mM diheptanoly thio-PC substrate, 0.29 mm Triton X-100™ detergent, and 0.12 mm DTMB in a buffered aqueous solution at pH 7.5.

Assay Procedure:

1. Add 0.2 ml reaction mixture to all wells;

2. Add 10 ul test compound (or solvent blank) to appropriate wells, mix 20 seconds;

3. Add 50 nanograms of sPLA₂ (10 microliters) to appropriate wells;

4. Incubate plate at 40° C. for 30 minutes;

5. Read absorbance of wells at 405 nanometers with an automatic plate reader.

All compounds were tested in triplicate. Typically, compounds were tested at a final concentration of 5 ug/ml. Compounds were considered active when they exhibited 40% inhibition or greater compared to uninhibited control reactions when measured at 405 nanometers. Lack of color development at 405 nanometers evidenced inhibition. Compounds initially found to be active were reassayed to confirm their activity and, if sufficiently active, IC₅₀ values were determined. Typically, the IC₅₀ values (see, Table I, below) were determined by diluting test compound serially two-fold such that the final concentration in the reaction ranged from 45 ug/mL to 0.35 ug/ml. More potent inhibitors required significantly greater dilution. In all cases, % inhibition measured at 405 nanometers generated by enzyme reactions containing inhibitors relative to the uninhibited control reactions was determined. Each sample was titrated in triplicate and result values were averaged for plotting and calculation of IC₅₀ values. IC₅₀ were determined by plotting log concentration versus inhibition values in the range from 10-90% inhibition.

Compounds of the instant invention were tested in Assay Example 1 and were found to be effective at concentrations of less than 100 μM.

Representative results are shown below:

For compounds of formula 2

2

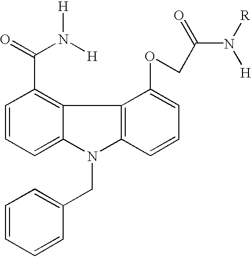

| | R | IC₅₀ (nM) |
|---|---|---|
| 2a | H | 151 |
| 2b | Et | 273 |

For compounds of formula 3

3

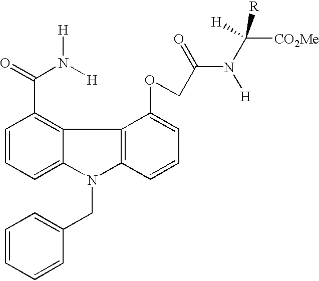

| | R | IC₅₀ (nM) |
|---|---|---|
| 3a | H | 383 |
| 3b | CH₃ | 205 |
| 3c | CH₂CH(CH₃)₂ | 67.5 |
| 3d | CH₂CO₂CH₃ | 513 |
| 3e | CH₂CH₂CO₂CH₃ | 709 |
| 3f | CH₂CH₂SCH₃ | 263 |
| 3g | CH₂Ph | 8600 |

For compounds of formula 4

4

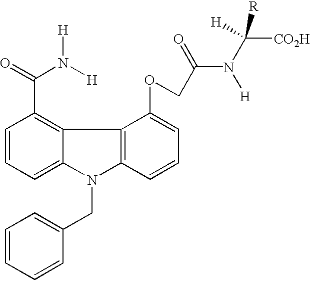

| | R | IC₅₀ (nM) |
|---|---|---|
| 4a | H | 118 |
| 4b | CH₃ | NA |
| 4c | CH₂CH(CH₃)₂ | 16.1 |
| 4d | CH₂CO₂H | 99.2 |
| 4e | CH₂CH₂CO₂H | 61.2 |
| 4f | CH₂CH₂SCH₃ | 103.4 |
| 4g | CH₂Ph | 324 |

While the present invention has been illustrated above by certain specific embodiments, it is not intended that these specific examples should limit the scope of the invention as described in the appended claims.

### EXAMPLES

### Example 1

[[5-Carbamoyl-9-(phenylmethyl)carbazol-4-yl]oxy]acetamide

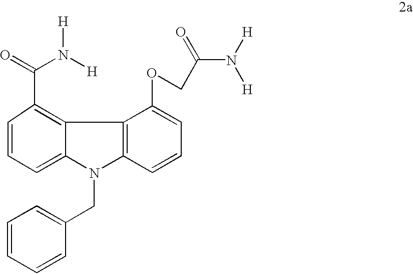

To a solution of 1 (0.200 g, 0.534 mmol) in 3 mL DMF was added collidine (0.078 mL, 0.587 mmol), then ammonia gas was bubbled into the reaction mixture, followed by the addition of benzotriazolyl-N-oxy-tris(dimethylamino)phosphonium hexafluorophosphate (0.248 g, 0.561 mmol). After 0.5 hr, 15 mL distilled H₂O were added to the reaction mixture and sonicated to give a white solid. The mixture was filtered, washed with distilled H₂O, and dried in a vacuum oven at 80° C. to give 0.152 g of 2a as white solid in 76% yield. ¹H NMR (DMSO-d₆) δ 4.58 (s, 2H), 5.66 (s, 2H), 6.60 (d, J=7.9 Hz, 1H), 7.07-7.41 (m, 10H), 7.52 (s, 1H), 7.62 (d, J=8.2 Hz, 1H), 7.79 (s, 1H), 8.01 (s, 1H).

### Example 2

[[5-Carbamoyl-9-(phenylmethyl)carbazol-4-yl]oxy]-N-(ethyl)acetamide

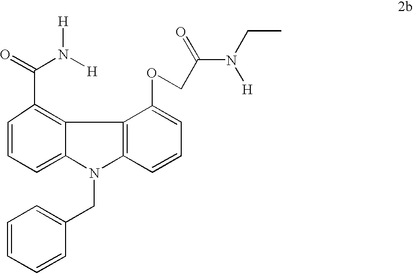

To a solution of 1 (0.150 g, 0.401 mmol) in 3 mL DMF was added collidine (0.058 mL, 0.441 mmol), then 2M ethylamine in THF (0.60, 1.20 mmol) was added to the reaction mixture at room temperature, followed by the addition of benzotriazolyl-N-oxy-tris(dimethylamino)phosphonium hexafluorophosphate (0.186 g, 0.421 mmol). After 1 hr the reaction mixture was concentrated in vacuo to near dryness, then it was taken up in CH₂Cl₂, chromatographed on a silica gel column (gradient 10-25% THF in CH₂Cl₂) and dried in an 80° C. vacuum oven to give 0.155 g of 2b as an off-white solid in 96% yield. ¹H NMR (CDCl₃) δ 1.17 (t, J=7.3 Hz, 3H), 3.38 (m, 2H), 4.75 (s, 2H), 5.53 (s, 2H), 5.89 (br s, 1H), 6.07 (br s, 1H), 6.71 (d, J=8.1 Hz, 1H), 7.06-7.11 (m, 3H), 7.23-7.46 (m, 7H), 8.05 (br s, 1H).

### Example 3

N-[[[5-Carbamoyl-9-(phenylmethyl)carbazol-4-yl]oxy]acetyl]glycine

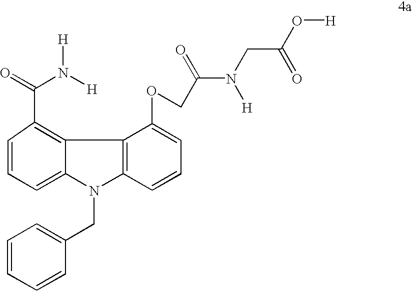

A. Preparation of N-[[[5-Carbamoyl-9-(phenylmethyl)carbazol-4-yl]oxy]acetyl]glycine methyl ester

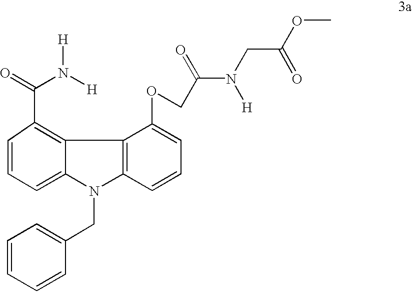

To a solution of 1 (0.200 g, 0.534 mmol) in 3 mL DMF was added collidine (0.148 mL, 1.12 mmol), methyl glycine hydrochloride (0.0671 g, 0.534 mmol), and benzotriazolyl-N-oxy-tris(dimethylamino)phosphonium hexafluorophosphate (0.248 g, 0.561 mmol) sequentially at room temperature. After 2 hrs. the reaction mixture was concentrated in vacuo to near dryness, then it was taken up in CH₂Cl₂ and chromatographed on a silica gel column (gradient 20-40% THF in CH₂Cl₂) to give 0.168 g of 3a as a white solid in 71% yield. ¹H NMR (DMSO-d₆) δ 3.60 (s, 3H), 3.98 (d, J=5.8 Hz, 2H), 4.70 (s, 2H), 5.67 (s, 2H), 6.76 (d, J=7.8 Hz, 1H), 7.07-7.46 (m, 10H), 7.65 (d, J=8.2 Hz, 1H), 8.15 (s, 1H), 9.04 (t, J=5.9 Hz, 1H).

A. Preparation of N-[[[5-Carbamoyl-9-(phenylmethyl)carbazol-4-yl]oxy]acetyl]glycine

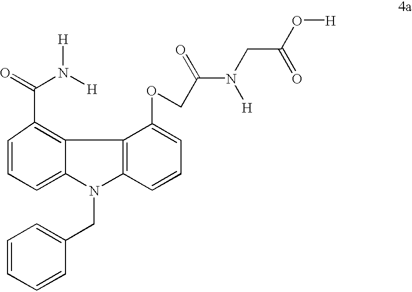

To a solution of 3a (0.0345 g, 0.078 mmol) in 1 mL THF, 1 mL MeOH and 0.25 mL distilled H₂O was added 4.17N LiOH (0.093 mL, 0.388 mmol) at room temperature. After 2 hrs. the reaction mixture was acidified with 5N HCl (0.093 mL, 0.465 mmol). The reaction mixture was diluted with 10 ml EtOAc, then 5 mL H₂O were added. The organic layer was separated from the aqueous layer and concentrated in vacuo and dried in an 80° C. vacuum oven to give 0.0292 g of 4a as a white solid in 87% yield. ¹H NMR (DMSO-d₆) δ 3.93 (d, J=5.8 Hz, 2H), 4.69 (s, 2H), 5.67 (s, 2H), 6.79 (d, J=7.8 Hz, 1H), 7.07-7.43 (m, 9H), 7.55 (s, 1H), 7.64 (d, J=8.2 Hz, 1H), 8.16 (s, 1H), 9.06 (t, J=5.8 Hz, 1H), 12.79 (br s, 1H).

### Example 4

N-[[[5-Carbamoyl-9-(phenylmethyl)carbazol-4-yl]oxy]acetyl]-L-alanine

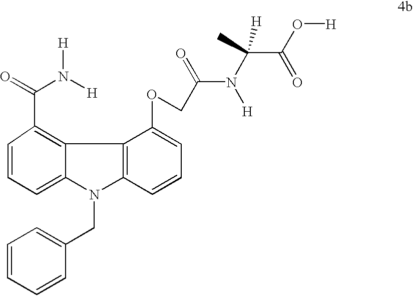

A. Preparation of N-[[[5-Carbamoyl-9-(phenylmethyl)carbazol-4-yl]oxy]acetyl]-L-alanine Methyl Ester

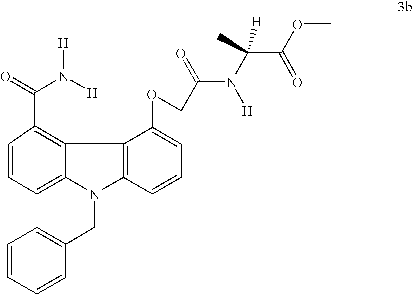

Following the experimental procedure as described for 3a, 3b was obtained as a white solid in 48% yield. ¹H NMR (DMSO-d₆) δ 1.33 (d, J=7.3 Hz, 3H), 3.56 (s, 3H), 4.33-4.38 (m, 1H), 4.58 (d, j=15.0 Hz, 1H), 4.78 (d, J=15.0 Hz, 1H), 5.67 (s, 1H), 6.73 (d, J=7.9 Hz, 1H), 7.06-7.43 (m, 9H), 7.50 (s, 1H), 7.64 (d, J=8.3 Hz, 1H), 8.19 (s, 1H), 8.99 (d, J=6.8 Hz, 1H).

B. Preparation of N-[[[5-Carbamoyl-9-(phenylmethyl)carbazol-4-yl]oxy]acetyl]-L-alanine

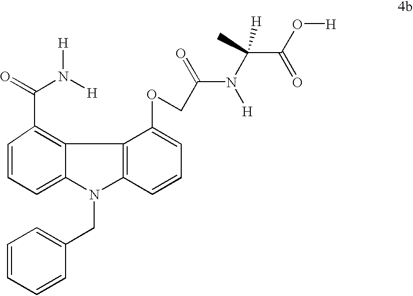

Following the experimental procedure as described for 4a, 4b was obtained as a white solid in % yield. ¹H NMR (DMSO-d₆) δ 1.36 (d, J=7.3 Hz, 3H), 4.27-4.34 (m, 1H), 4.48 (d, J=14.8 Hz, 1H), 4.82 (d, J=14.8 Hz, 1H), 5.67 (s, 2H), 6.79 (d, J=7.8 Hz, 1H), 7.02-7.42 (m, 9H), 7.63 (d, J=8.2 Hz, 1H), 7.88 (br s, 1H), 8.17 (s, 1H), 9.07 (d, J=7.2 Hz, 1H), 12.25-12.70 (br s, 1H).

### Example 5

N-[[[5-Carbamoyl-9-(phenylmethyl)carbazol-4-yl]oxy]acetyl]-L-leucine

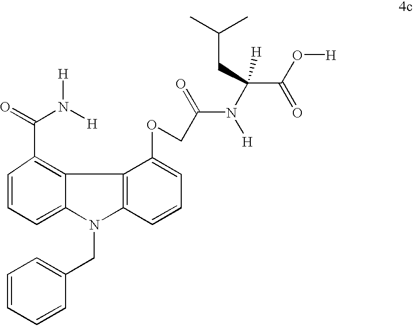

A. Preparation of N-[[[5-Carbamoyl-9-(phenylmethyl)carbazol-4-yl]oxy]acetyl]-L-leucine Methyl Ester

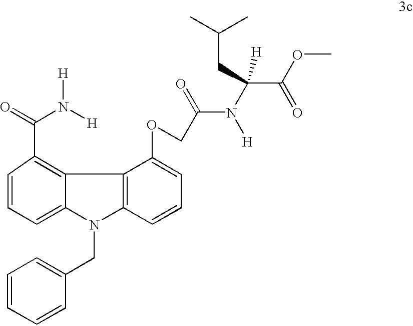

Following the experimental procedure as described for 3a, 3c was obtained as a white solid in 80% yield. ¹H NMR (DMSO-d₆) δ 0.61 (d, J=5.5 Hz, 6H), 1.29-1.45 (m, 2H), 1.57-1.66 (m, 1H), 3.54 (s, 3H), 4.22-4.29 (m, 1H), 4.58 (d, J=14.9 Hz, 1H), 4.84 (d, J=14.9 Hz, 1H), 5.67 (s, 2H), 6.70 (d, J=7.8 Hz, 1H), 7.06-7.42 (m, 9H), 7.56 (s, 1H), 7.63 (d, J=8.2 Hz, 1H), 8.22 (s, 1H), 8.93 (d, J=72 Hz, 1H); ESIMS m/e (M⁺+1).

B. Preparation of N-[[[5-Carbamoyl-9-(phenylmethyl)carbazol-4-yl]oxy]acetyl]-L-leucine

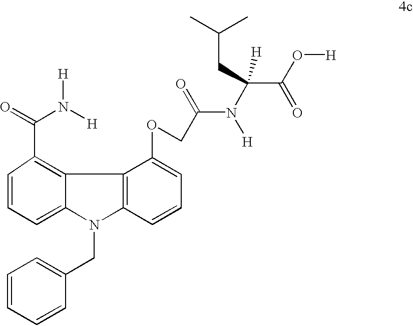

Following the experimental procedure as described for 4a, 4c was obtained as a white solid in 97% yield. ¹H NMR (DMSO-d₆) δ 0.66 (d, J=5.0 Hz, 6H), 1.44-1.51 (m, 2H), 1.69 (m, 1H), 4.22-4.27 (m, 1H), 4.53 (d, J=14.9 Hz, 1H), 4.87 (d, J=14.9 Hz, 1H), 5.68 (s, 2H), 6.76 (d, J=7.8 Hz, 1H), 7.06-7.42 (m, 9H), 7.62 (s, 1H), 7.66 (d, J=7.4 Hz;-1H), 8.23 (s, 1H), 9.01 (d, J=7.6 Hz, 1H), 12.84 (br s, 1H); ESIMS m/e (M+1).

### Example 6

N-[[[5-Carbamoyl-9-(phenylmethyl)carbazol-4-yl]oxy]acetyl]-L-aspartic Acid

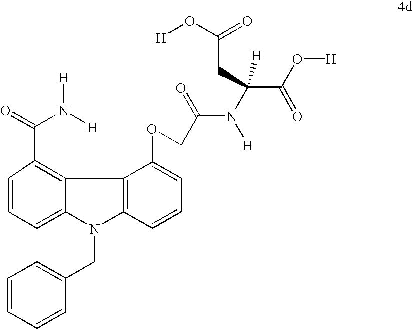

A. Preparation of N-[[[5-Carbamoyl-9-(phenylmethyl)carbazol-4-yl]oxy]acetyl]-L-aspartic Acid Dimethyl Ester

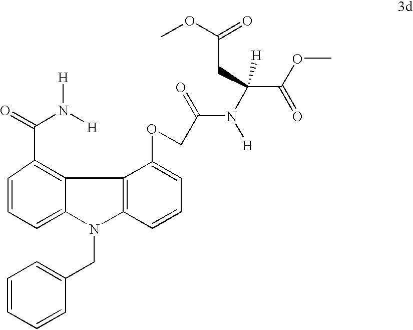

Following the experimental procedure as described for 3a, 3d was obtained as a white solid in 70% yield. ¹H NMR (DMSO-d₆) δ 2.80-2.84 (m, 2H), 3.43 (s, 3H), 3.54 (s, 3H), 4.62-4.77 (m, 3H), 5.67 (s, 2H), 6.71 (d, J=7.9 Hz, 1H), 7.06-7.43 (m, 10H), 7.63 (d, J=8.2 Hz, 1H), 8.12 (s, 1H), 8.99 (d, J=7.6 Hz, 1H).

B. Preparation of N-[[[5-Carbamoyl-9-(phenylmethyl)carbazol-4-yl]oxy]acetyl]-L-aspartic Acid

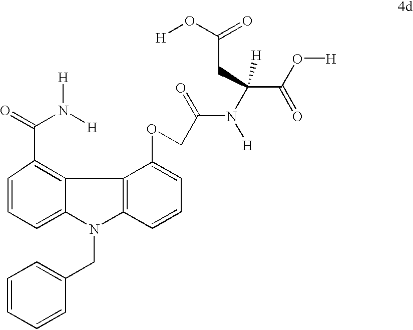

Following the experimental procedure as described for 4a, 4d was obtained as a white solid in 94% yield. ¹H NMR (DMSO-d₆) δ 2.68-2.85 (m, 2H), 4.57 (d, J=15.0 Hz, 1H), 4.64-4.71 (m, 1H), 4.77 (d, J=15.0 Hz, 1H), 5.67 (s, 2H), 6.79 (d, J=7.8 Hz, 1H), 7.06-7.42 (m, 9H), 7.48 (s, 1H), 7.63 (d, J=8.3 Hz, 1H), 8.17 (s, 1H), 9.06 (d, J=7.9 Hz, 1H), 12.6-13.2 (br s, 2H).

### Example 7

N-[[[5-Carbamoyl-9-(phenylmethyl)carbazol-4-yl]oxy]acetyl]-L-glutamic Acid

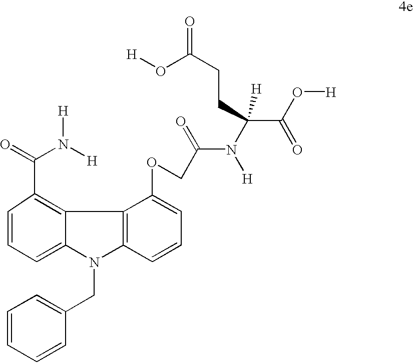

B. Preparation of N-[[[5-Carbamoyl-9-(phenylmethyl)carbazol-4-yl]oxy]acetyl]-L-glutamic Acid Dimethyl Ester

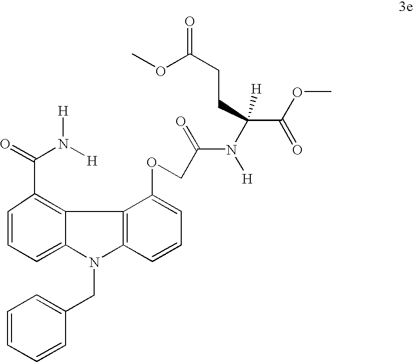

Following the experimental procedure as described for 3a, 3e was obtained as a white solid in 85% yield. ¹H NMR (DMSO-d₆) δ 1.83-2.02 (m, 2H), 2.20-2.28 (m, 2H), 3.46 (s, 3H), 3.53 (s, 3H), 4.28-4.35 (m, 1H), 4.60 (d, J=15.0 Hz, 1H), 4.80 (d, J=15.0 Hz, 1H), 5.67 (s, 2H), 6.69 (d, J=7.9 Hz, 1H), 7.06-7.42 (m, 9H), 7.53 (s, 1H), 7.63 (d, J=8.2 Hz, 1H), 8.20 (s, 1H), 8.93 (d, J=7.3 Hz, 1H)

B. Preparation of N-[[[5-Carbamoyl-9-(phenylmethyl)carbazol-4-yl]oxy]acetyl]-L-glutamic Acid

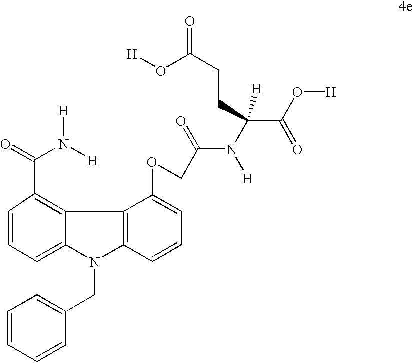

Following the experimental procedure as described for 4a, 4e was obtained as a white solid in 87% yield. ¹H NMR (DMSO-d₆) δ 1.87-2.06 (m, 2H), 2.24 (t, J=7.5 Hz, 2H), 4.29-4.36 (m, 1H), 4.53 (d, J=14.9 Hz, 1H), 4.86 (d, J=14.90 Hz, 1H), 5.67 (s, 2H), 6.76 (d, J=7.8 Hz, 1H), 7.06-7.42 (m, 9H), 7.63 (d, J=8.2 Hz, 1H), 7.66 (s, 1H), 8.20 (s, 1H), 9.02 (d, J=7.6 Hz, 1H), 12.2-12.9 (br s, 2H).

### Example 8

N-[[[5-Carbamoyl-9-(phenylmethyl)carbazol-4-yl]oxy]acetyl]-L-methionine

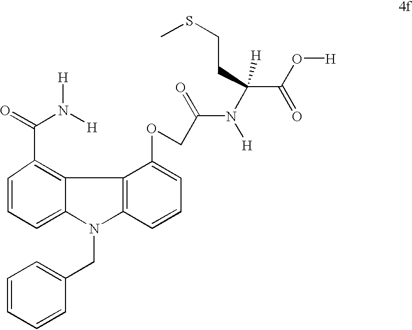

A. Preparation of N-[[[5-Carbamoyl-9-(phenylmethyl)carbazol-4-yl]oxy]acetyl]-L-methionine Methyl Ester

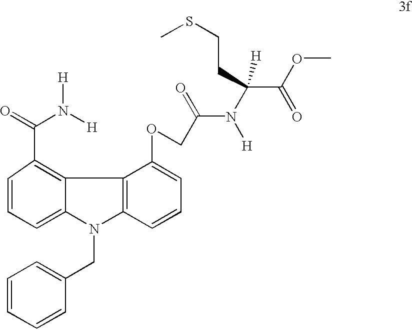

Following the experimental procedure as described for 3a, 3f was obtained as a white solid in 92% yield. ¹H NMR (DMSO-d₆) δ 1.76 (s, 3H), 1.83-1.93 (m, 2H), 2.19-2.28 (m, 2H), 3.55 (s, 3H), 4.36-4.43 (m, 1H), 4.63 (d, J=15.0 Hz, 1H), 4.81 (d, J=15.0 Hz, 1H), 5.67 (s, 2H), 6.68 (d, J=7.8 Hz, 1H), 7.06-7.42 (m, 9H), 7.59-7.64 (m 2H), 8.21 (s, 1H), 8.91 (d, J=7.4 Hz, 1H).

B. Preparation of N-[[[5-Carbamoyl-9-(phenylmethyl)carbazol-4-yl]oxy]acetyl]-L-methionine

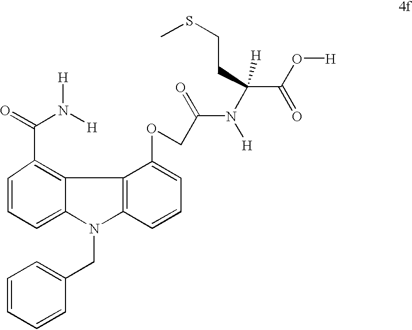

Following the experimental procedure as described for 4a, 4f was obtained as a white solid in 95% yield. ¹H NMR (DMSO-d₆) δ 1.80 (s, 3H), 1.89-1.97 (M, 2H), 2.20-2.31 (m, 2H), 4.33-4.39 (m, 1H), 4.57 (d, J=14.9 Hz, 1H), 4.83 (d, J=14.9 Hz, 1H), 5.67 (s, 2H), 6.73 (d, J=7.8 Hz, 1H), 7.05-7.42 (m, 9H), 7.59-7.64 (m 2H), 8.21 (s, 1H), 8.91 (d, J=7.4 Hz, 1H), 12.7-13.2 (br s, 1H).

### Example 9

N-[[[5-Carbamoyl-9-(phenylmethyl)carbazol-4-yl]oxy]acetyl]-L-phenylalanine

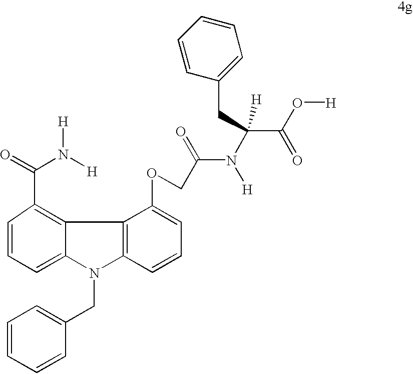

A. Preparation of N-[[[5-Carbamoyl-9-(phenylmethyl)carbazol-4-yl]oxy]acetyl]-L-phenylalanine Methyl Ester

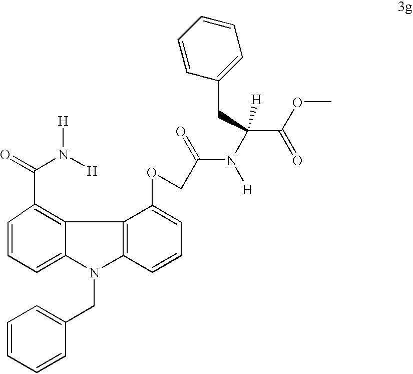

Following the experimental procedure as described for 3a, 3 g was obtained as a white solid in 95% yield. ¹H NMR (DMSO-d₆) δ 3.01-3.04 (m, 2H), 3.56 (s, 3H), 4.43-4.52 (m, 1H), 4.53 (d, J=15.0 Hz, 1H), 4.74 (d, J=15.0 Hz, 1H), 5.68 (s, 2H), 6.61 (d, J=6.6 Hz, 1H), 7.01-7.50 (m, 14H), 7.53 (s, 1H), 7.67 (d, J=8.3 Hz, 1H), 8.24 (s, 1H), 9.11 (d, J=7.6 Hz, 1H).

B. Preparation of N-[[[5-Carbamoyl-9-(phenylmethyl)carbazol-4-yl]oxy]acetyl]-L-phenylalanine

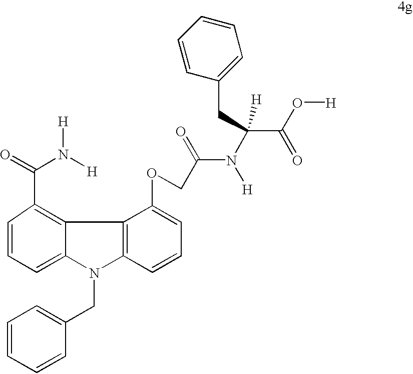

Following the experimental procedure as described for 4a, 4 g was obtained as a white solid in 99% yield. ¹H NMR (DMSO-d₆) δ 3.05-3.12 (m, 2H), 4.42-4.47 (m, 1H), 4.44 (d, J=14.9 Hz, 1H), 4.75 (d, J=14.9 Hz, 1H), 5.68 (s, 2H), 6.66 (d, J=6.4 Hz, 1H), 7.03-7.33 (m, 13H), 7.39-7.44 (m, 1H), 7.66 (d, J=8.3 Hz, 1H), 7.73 (s, 1H), 8.24 (s, 1H), 9.17 (d, J=7.3 Hz, 1H), 13.01-13.24 (br s, 1H).

We claim:

1. A compound of the formula (I) 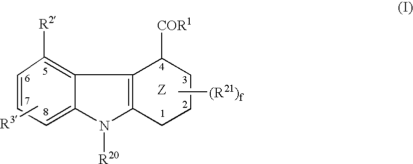
wherein;
Z is cyclohexenyl, or phenyl,
R⁴ is selected from groups (a), (b) and (c) where;
(a) is —(C₅-C₂₀)alkyl, —(C₅-C₂₀)alkenyl, —(C₅-C₂₀)alkynyl, carbocyclic radicals, or heterocyclic radicals, or
(b) is a member of (a) substituted with one or more independently selected non-interfering substituents; or
(c) is the group —(L)—R⁸⁰; where, (L)-is a divalent linking group of 1 to 12 atoms selected from carbon, hydrogen, oxygen, nitrogen, and sulfur; wherein the combination of atoms in —(L)— are selected from the group consisting of (i) carbon and hydrogen only, (ii) one sulfur only, (iii) one oxygen only, (iv) one or two nitrogen and hydrogen only, (v) carbon, hydrogen, and one sulfur only, and (vi) an carbon, hydrogen, and oxygen only; and where R⁸⁰ is a group selected from (a) or (b);
R²¹ is a non-interfering substituent where f is 1-3;
R¹ is —NHNH₂, —NH₂, or —CONH₂;
R², is the group
—O(CH₂)ₜR⁵, where
R^{5′} is —CONR⁹R¹⁰; wherein R⁹ and R¹⁰ are independently hydrogen, (C₁-C₆)alkyl, phenyl, or phenyl substituted with —CO₂H or —CO₂ (C₁-C₄) alkyl where m is 1-3; or
(a) or
(b) R^{5′} is the group —(Lₕ)-(acylamino acid), wherein —(Lₕ)— is an acylamino acid linker having an acylamino acid linker length of 1 to 7 and t is 1-5;
R^{3′} is selected from non-interfering substituent, carbocyclic radicals, carbocyclic radicals substituted with non-interfering substituents, heterocyclic radicals, and heterocyclic radicals substituted with non-interfering substituents; or a pharmaceutically acceptable racemate, solvate, tautomer, optical isomer, prodrug derivative or salt, thereof.

2. A compound of the formula (II) 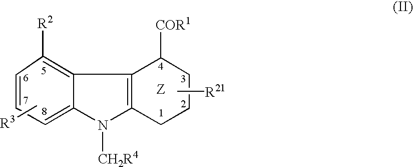
wherein;
Z is cyclohexenyl, or phenyl;
R²¹ is a non-interfering substituent;
R¹ is —NHNH₂, —NH₂, or CONH₂;
R₂ is the group
—O(CH₂)ₘR⁵, where
R⁵ is
(a) —CONR⁹R¹⁰, wherein R⁹ and R¹⁰ are independently hydrogen, (C₁-C₆)alkyl, phenyl, or phenyl substituted with —CO₂H or —CO₂(C₁-C₄)alkyl where m is 1-3; or
(b) —(Lₕ)-(acylamino acid), wherein —(Lₕ)— is an acylamino acid linker having an acylamino acid linker length of 1 to 7;
R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙR⁸ where R⁸ is H, —CONH₂, —NR⁹R¹⁰, —CN or phenyl where R⁹ and R¹⁰ are independently —(C₁-C₄)alkyl or -phenyl(C₁-C₄)alkyl and n is 1 to 8;
R⁴ is H, —(C₁-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy or naphthyl;
or a pharmaceutically acceptable racemate, solvate, tautomer, optical isomer, prodrug derivative or salt, thereof.

3. A compound according to claim 1 wherein R¹ is —CONH₂, —NH₂ or NHNH₂; and Z is phenyl.

4. A pharmaceutical formulation comprising a compound of formula I as claimed in claim 1 together with a pharmaceutically acceptable carrier or diluent therefor.

5. A pharmaceutical formulation comprising a compound of formula II as claimed in claim 2 together with a pharmaceutically acceptable carrier or diluent therefor.

6. A pharmaceutical formulation adapted for the treatment of a condition associated with inhibiting sPLA₂, containing a compound of formula I as claimed in claim 1 together with a pharmaceutically acceptable carrier or diluent therefor.

7. A pharmaceutical formulation adapted for the treatment of a condition associated with inhibiting sPLA₂, containing a compound of formula II as claimed in claim 2 together with a pharmaceutically acceptable carrier or diluent therefor.

8. A method of selectively inhibiting sPLA₂ in a mammal in need of such treatment comprising administering to said mammal a therapeutically effective amount of a compound of formula (I) 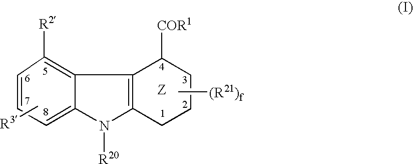
wherein;
z is cyclohexenyl, or phenyl,
R⁴ is selected from groups (a), (b) and (c) where;
(a) is —(C₅-C₂₀)alkyl, —(C₅-C₂₀)alkenyl, —(C₅-C₂₀)alkynyl, carbocyclic radicals, or heterocyclic radicals, or
(b) is a member of (a) substituted with one or more independently selected non-interfering substituents; or
(c) is the group —(L)—R⁸⁰; where, (L)-is a divalent linking group of 1 to 12 atoms selected from carbon, hydrogen, oxygen, nitrogen, and sulfur; wherein the combination of atoms in —(L)— are selected from the group consisting of (i) carbon and hydrogen only, (ii) one sulfur only, (iii) one oxygen only, (iv) one or two nitrogen and hydrogen only, (v) carbon, hydrogen, and one sulfur only, and (vi) an carbon, hydrogen, and oxygen only; and where R⁸⁰ is a group selected from (a) or (b);
R²¹ is a non-interfering substituent where f is 1-3;
R¹ is —NHNH₂, —NH₂, or —CONH₂;
R², is the group
—O(CH₂)ₜR^{5′} where
(a) R^{5′} is —CONR⁹R¹⁰; wherein R⁹ and R¹⁰ are independently hydrogen, (C₁-C₆)alkyl, phenyl, or phenyl substituted with —CO₂H or —CO₂(C₁-C₄)alkyl; or
(b) R^{5′} is the group —(Lₕ)-(acylamino acid), wherein —(Lₕ)— is an acylamino acid linker having an acylamino acid linker length of 1 to 7 and t is 1-5;
R^{3′} is selected from non-interfering substituent, carbocyclic radicals, carbocyclic radicals substituted with non-interfering substituents, heterocyclic radicals, and heterocyclic radicals substituted with non-interfering substituents;
or a pharmaceutically acceptable racemate, solvate, tautomer, optical isomer, prodrug derivative or salt, thereof.

9. A method of selectively inhibiting sPLA₂ in a mammal in need of such treatment comprising administering to said mammal a therapeutically effective amount of a compound of formula (IIa) 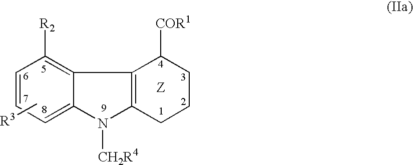
wherein;
R¹ is —CONH₂, —NHNH₂, or —NH₂;
R² is the group
—O(CH₂)ₘR⁵ where
(a) R⁵ is —CONR⁹R¹⁰; wherein R⁹ and R¹⁰ are independently hydrogen, (C₁-C₆)alkyl, phenyl, or phenyl substituted with —CO₂H or —CO₂(C₁′-C₄)alkyl; or
R⁵ is the group —(Lₕ)-(acylamino acid), wherein —(Lₕ)— is an acylamino acid linker having an acylamino acid linker length of 1 to 7 and m is 1-3;
R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙR⁸ where R⁸ is H, —CONH₂, —NR⁹R¹⁰, —CN or phenyl where R⁹ and R¹⁰ are independently —(C₁-C₄)alkyl or -phenyl(C₁-C₄)alkyl and n is 1 to 8;
R⁴ is H, —(C₁-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy or naphthyl;
Z is cyclohexenyl, or phenyl;
or a pharmaceutically acceptable racemate, solvate, tautomer, optical isomer, prodrug derivative or salt, thereof.

10. A method of claim 10 wherein the mammal is a human.

11. A method of claim 13 wherein the mammal is a human.

12. A method of alleviating the pathological effects of sPLA₂ related diseases which comprises administering to a mammal in need of such treatment a compound of formula I as claimed in claim 1 in an amount sufficient to inhibit sPLA₂ mediated release of fatty acid and to thereby inhibit or prevent the arachidonic acid cascade and its deleterious products.

13. A method of alleviating the pathological effects of sPLA₂ related diseases which comprises administering to a mammal in need of such treatment a compound of formula II as claimed in claim 2 in an amount sufficient to inhibit sPLA₂ mediated release of fatty acid and to thereby inhibit or prevent the arachidonic acid cascade and its deleterious products.

14. The use of a compound of formula I as claimed in claim 1 for the manufacture of a medicament for alleviating the pathological effects of sPLA₂ related diseases which comprises administering to a mammal in need of such treatment a compound of formula I.

15. A method of inhibiting sPLA₂ which comprises contacting the sPLA₂ with a compound of formula I as claimed in claim 1.

16. A method of inhibiting sPLA₂ which comprises contacting the sPLA₂with a compound of formula II as claimed in claim 2.

17. A method of treating sepsis, septic shock, rheumatoid arthritis, osteoarthritis, stroke, apoptosis, asthma, chronic bronchitis, acute bronchitis, cystic fibrosis, inflammatory bowel disease, or pancreatitis which comprises administering to a mammal in need of such treatment, a therapeutically effective amount of a compound of formula I 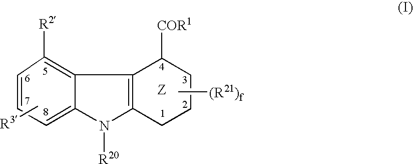
wherein;
Z is cyclohexenyl, or phenyl,
R⁴ is selected from groups (a), (b) and (c) where;
(a) is —(C₅-C₂₀)alkyl, —(C₅-C₂₀)alkenyl, —(C₅-C₂₀)alkynyl, carbocyclic radicals, or heterocyclic radicals, or
(b) is a member of (a) substituted with one or more independently selected non-interfering substituents; or
(c) is the group —(L)—R⁸⁰; where, (L)-is a divalent linking group of 1 to 12 atoms selected from carbon, hydrogen, oxygen, nitrogen, and sulfur; wherein the combination of atoms in —(L)— are selected from the group consisting of (i) carbon and hydrogen only, (ii) one sulfur only, (iii) one oxygen only, (iv) one or two nitrogen and hydrogen only, (v) carbon, hydrogen, and one sulfur only, and (vi) an carbon, hydrogen, and oxygen only; and where R⁸⁰ is a group selected from (a) or (b);
R^{2′} is a non-interfering substituent where f is 1-3;
R¹ is —NHNH₂, —NH₂, or —CONH₂;
R^{2′} is the group —O(CH₂)ₜR^{5′} where
R^{5′} is (a) —CONR⁹R¹⁰ where R⁹ and R¹⁰ are independently Hydrogen, —(C₁-C₆)alkyl or —CF₃; phenyl or phenyl substituted with —(C₁-C₆)alkyl; or
(b) —(Lₕ)-(acylamino acid) group, wherein —(Lₕ)— is an acylamino acid linker having an “acylamino acid” linker length of 1 to 7 and t is 1-5;
R³, is selected from non-interfering substituent, carbocyclic radicals, carbocyclic radicals substituted with non-interfering substituents, heterocyclic radicals, and heterocyclic radicals substituted with non-interfering substituents;
or a pharmaceutically acceptable racemate, solvate, tautomer, optical isomer, prodrug derivative or salt, thereof.

18. A method of treating sepsis, septic shock, rheumatoid arthritis, osteoarthritis, stroke, apoptosis, asthma, chronic bronchitis, acute bronchitis, cystic fibrosis, inflammatory bowel disease, or pancreatitis which comprises administering to a subject in need of such treatment, a therapeutically effective amount of a compound of formula II 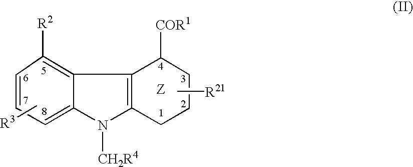
wherein;
Z is cyclohexenyl, or phenyl,
R²¹ is a non-interfering substituent;
R¹ is —CONH₂, —NHNH₂ or —NH₂;
R^{2′} is the group
—O(CH₂)ₜR^{5′} where
(a) R^{5′} is —CONR⁹R¹⁰; wherein R⁹ and R¹⁰ are independently hydrogen, (C₁-C₆)alkyl, phenyl, or phenyl substituted with —CO₂H or —CO₂(C₁-C₄)alkyl; or
(b) R^{5′} is the group —(Lₕ)-(acylamino acid), wherein —(Lₕ)— is an acylamino acid linker having an acylamino acid linker length of 1 to 7 and t is 1-5;
R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi(C₁-C₆)alkyl, furyl, thiophenyl, —(C₁-C₆)hydroxyalkyl; or —(CH₂)ₙR⁸ where R⁸ is H, —CONH₂, —NR⁹R¹⁰, —CN or phenyl where R⁹ and R¹⁰ are independently —(C₁-C₄)alkyl or -phenyl(C₁-C₄)alkyl and n is 1 to 8;
R⁴ is H, —(C₁-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy or naphthyl;
or a pharmaceutically acceptable racemate, solvate, tautomer, optical isomer, prodrug derivative or salt, thereof.

19. A method of claim 10 of alleviating the pathological effects of sepsis, septic shock, adult respiratory distress syndrome, pancreatitis, trauma-induced shock, bronchial asthma, allergic rhinitis, rheumatoid arthritis, cystic fibrosis, stroke, acute bronchitis, chronic bronchitis, acute bronchiolitis, chronic bronchiolitis, osteoarthritis, gout, spondylarthropathris, ankylosing spondylitis, Reiter's syndrome, psoriatic arthropathy, enterapathric spondylitis, Juvenile arthropathy or juvenile ankylosing spondylitis, Reactive arthropathy, infectious or post-infectious arthritis, gonoccocal arthritis, Tuberculous arthritis, viral arthritis, fungal arthritis, syphilitic arthritis, Lyme disease, arthritis associated with “vasculitic syndromes”, polyarteritis nodosa, hypersensitivity vasculitis, Luegenec's. granulomatosis, polymyalgin rheumatica, joint cell arteritis, calcium crystal deposition arthropathris, pseudo gout, non-articular rheumatism, bursitis, tenosynomitis, epicondylitis (tennis elbow), carpal tunnel syndrome, repetitive use injury (typing), miscellaneous forms of arthritis, neuropathic joint disease (charco and joint), hemarthrosis (hemarthrosic), Henoch-Schonlein Purpura, hypertrophic osteoarthropathy, multicentric reticulohistiocytosis, arthritis associated with certain diseases, surcoilosis, hemochromatosis, sickle cell disease and other hemoglobinopathries, hyperlipoproteineimia, hypogammaglobulinemia, hyperparathyroidism, acromegaly, familial Mediterranean fever, Behat's Disease, systemic lupus erythrematosis, or relapsing polychondritis; and related diseases which comprises administering to a mammal in need of such treatment a therapeutically effective amount of a compound of formula I.

20. A method of claim 11 of alleviating the pathological effects of sepsis, septic shock, adult respiratory distress syndrome, pancreatitis, trauma-induced shock, bronchial asthma, allergic rhinitis, rheumatoid arthritis, cystic fibrosis, stroke, acute bronchitis, chronic bronchitis, acute bronchiolitis, chronic bronchiolitis, osteoarthritis, gout, spondylarthropathris, ankylosing spondylitis, Reiter's syndrome, psoriatic arthropathy, enterapathric spondylitis, Juvenile arthropathy or juvenile ankylosing spondylitis, Reactive arthropathy, infectious or post-infectious arthritis, gonoccocal arthritis, Tuberculous arthritis, viral arthritis, fungal arthritis, syphilitic arthritis, Lyme disease, arthritis associated with “vasculitic syndromes”, polyarteritis nodosa, hypersensitivity vasculitis, Luegenec's granulomatosis, polymyalgin rheumatica, joint cell arteritis, calcium crystal deposition arthropathris, pseudo gout, non-articular rheumatism, bursitis, tenosynomitis, epicondylitis (tennis elbow), carpal tunnel syndrome, repetitive use injury (typing), miscellaneous forms of arthritis, neuropathic joint disease (charco and joint), hemarthrosis (hemarthrosic), Henoch-Schonlein Purpura, hypertrophic osteoarthropathy, multicentric reticulohistiocytosis, arthritis associated with certain diseases, surcoilosis, hemochromatosis, sickle cell disease and other hemoglobinopathries, hyperlipoproteineimia, hypogammaglobulinemia, hyperparathyroidism, acromegaly, familial Mediterranean fever, Behat's Disease, systemic lupus erythrematosis, or relapsing polychondritis;
and related diseases which comprises administering to a mammal in need of such treatment a therapeutically effective amount of a compound of formula II.

21. A process of preparing compounds of formula II 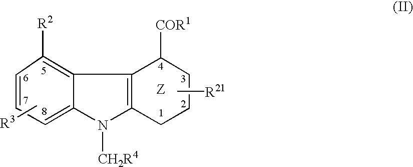
wherein;
Z is cyclohexenyl, or phenyl,
R²¹ is a non-interfering substituent;
R¹ is —NHNH₂ or —NH₂;
R² is the group —O(CH₂)ₘR⁵ where
(a) R⁵ is —CONR⁹R¹⁰; wherein R⁹ and R¹⁰ are independently hydrogen, (C₁-C₆)alkyl, phenyl, or phenyl substituted with —CO₂H or —CO₂(C₁-C₄)alkyl; or
(b) R⁵ is the group —(Lₕ)-(acylamino acid), wherein —(Lₕ)— is an acylamino acid linker having an acylamino acid linker length of 1 to 7; and where m is 1-3;
R³ is H, —O(C₁-C₄)alkyl, halo, —(C₁-C₆)alkyl, phenyl, —(C₁-C₄)alkylphenyl; phenyl substituted with —(C₁-C₆)alkyl, halo, or —CF₃; —CH₂OSi (C₁-C₆) alkyl, furyl, thiophenyl, —(C₁-C₆)hydroxyalkyl; or —(CH₂)ₙR⁸ where R⁸ is H, —CONH₂, —NR⁹R¹⁰, —CN or phenyl where R⁹ and R¹⁰ are independently —(C₁-C₄)alkyl or -phenyl(C₁-C₄)alkyl and n is 1 to 8; R⁴ is H, —(C₁-C₁₄)alkyl, —(C₃-C₁₄)cycloalkyl, pyridyl, phenyl or phenyl substituted with —(C₁-C₆)alkyl, halo, —CF₃₁—OCF3, —(C₁-C₄)alkoxy, —CN, —(C₁-C₄)alkylthio, phenyl (C₁-C₄) alkyl, —(C₁-C₄) alkylphenyl, phenyl, phenoxy or naphthyl;
or a pharmaceutically acceptable racemate, solvate, tautomer, optical isomer, prodrug derivative or salt, thereof;
a) esterifying a compound of formula XVI 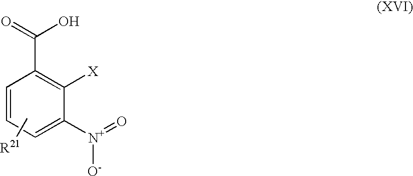
where X is halo;
to form a compound of formula XV 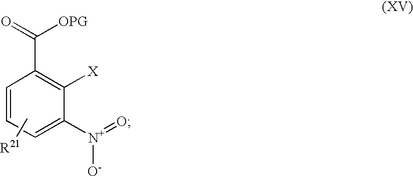
b) reducing a compound of formula XV to form a compound of formula XIV 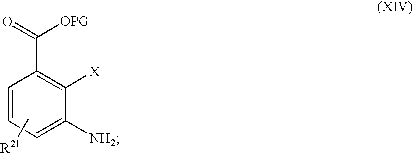
where PG is an acid protecting group
c) condensing a compound of formula XIV with a compound of formula XIII 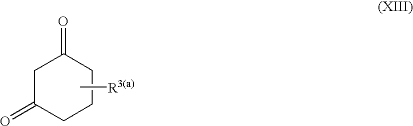
where R³ (a) is H, —O(C₁-C₄)alkyl, halo, —(C₁-C₆)alkyl, phenyl, —(C₁-C₄)alkylphenyl; phenyl substituted with —(C₁-C₆)alkyl, halo or —CF₃; —CH₂OSi (C₁-C₆) alkyl, furyl, thiophenyl, —(C₁-C₆)hydroxyalkyl; or —(CH₂)ₙR⁸ where R⁸ is H, —NR 9R¹⁰, —CN or phenyl where R⁹ and R¹⁰ are independently —(C₁-C₄)alkyl or -phenyl(C₁-C₄)alkyl and n is 1 to 8;
to form a compound of formula XII 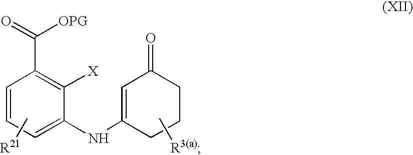
d) cyclizing a compound of formula XII to form a compound of formula VI 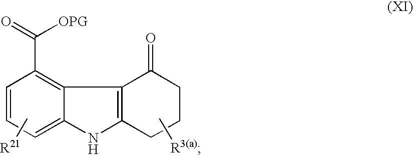
e) alkylating a compound of formula XI
with an alkylating agent of the formula XCH₂R⁴, where X is halo to form a compound of formula X 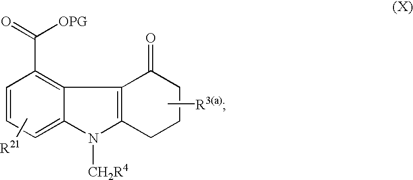
f) dehydrogenating a compound of formula X to form a compound of formula IX 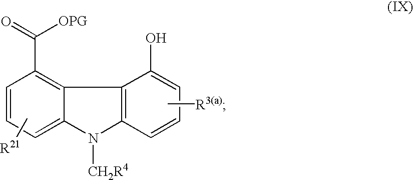
g) aminating a compound of formula IX to form a compound of formula VIII 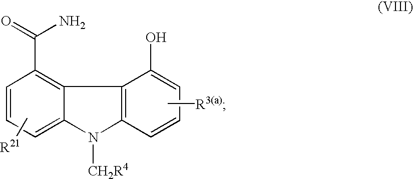
h) alkylating a compound of formula VIII with an alkylating agent of formula XCH₂R¹⁵ where X is halo and R¹⁵ is —CO₂R¹⁶, —SO₃R¹⁶, —P(O)(OR ¹⁶)₂, or —P(O) (OR¹⁶)H, where R¹⁶ is an acid protecting group to form a compound of formula VII 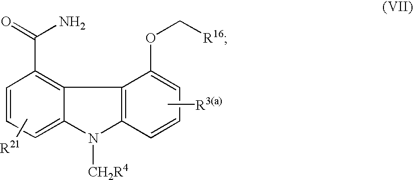
i) hydrolyzing a compound of formula VII to form a compound of formula I; and
j) converting a compound of formula VII to a compound of formula I.

22. A process for preparing compounds of formula II, 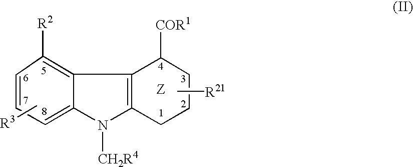
wherein;
Z is cyclohexenyl, or phenyl, R^{21′} is a non-interfering substituent;
R¹ is —CONH₂, —NHNH₂ or —NH₂;
R² is the group
—O(CH₂)ₜR⁵ where
(a) R⁵ is —CONR⁹R¹⁰; wherein R⁹ and R¹⁰ are independently hydrogen, (C₁-C₆)alkyl, phenyl, or phenyl substituted with —CO₂H or —CO₂(C₁-C₄)alkyl; or
(b) R⁵ is the group —(Lₕ)-(acylamino acid), wherein —(Lₕ)— is an acylamino acid linker having an acylamino acid linker length of 1 to 7 and where m is 1-3;
R³ is H, —O(C₁-C₄)alkyl, halo, —(C₁-C₆)alkyl, phenyl, —(C₁-C₄) alkylphenyl; phenyl substituted with —(C₁-C₆) alkyl, halo, or —CF₃; —CH₂OSi(C₁-C₆)alkyl, furyl, thiophenyl, —(C₁-C₆)hydroxyalkyl; or —(CH₂)ₙR⁸ where R⁸ is H, —CONH₂, —NR⁹R¹⁰, —CN or phenyl where R⁹ and R¹⁰ are independently —(C₁-C₄)alkyl or -phenyl(C₁-C₄)alkyl and n is 1 to 8;
R⁴ is H, —(C₁-C₁₄)alkyl, —(C₃-C₁₄)cycloalkyl, pyridyl, phenyl or phenyl substituted with —(C₁-C₆)alkyl, halo, —CF₃, —OCF₃, —(C₁-C₄)alkoxy, —CN, —(C₁-C₄)alkylthio, phenyl (C₁-C₄) alkyl, —(C₁-C₄) alkylphenyl, phenyl, phenoxy or naphthyl;
or a pharmaceutically acceptable racemate, solvate, tautomer, optical isomer, prodrug derivative or salt, thereof which process comprises the steps of:
a) esterifying a compound of formula XVI 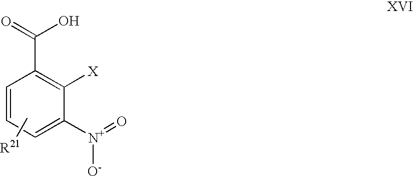
where X is halo to form a compound of formula XV 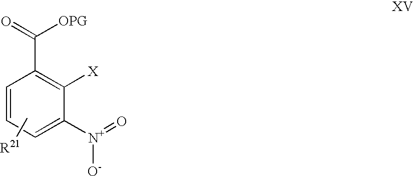
where PG is an acid protecting group;
b) condensing a compound of formula XV with a compound of formula XVII 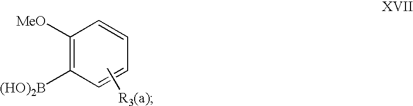
to form a compound of formula XVIII 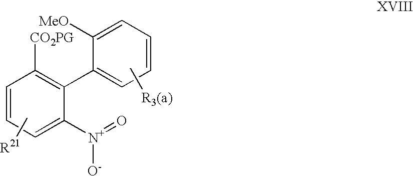
c) cyclizing a compound of formula XVIII to form a compound of formula XIX. 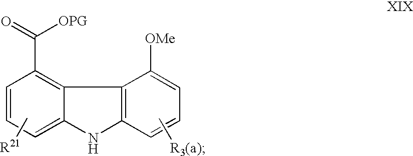
d) alkylating a compound of formula XIX with an alkylating agent of the formula XCH₂R⁴, where X is halo, to form a compound of formula XX 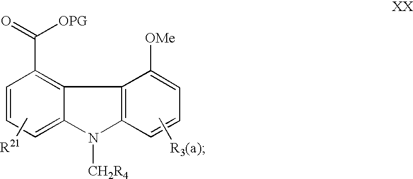
e) dealkylating a compound of formula XX to form a compound of formula IX 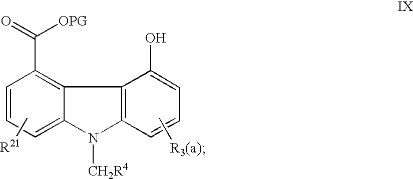
f) aminating compound of formula IX to form a compound of formula VIII 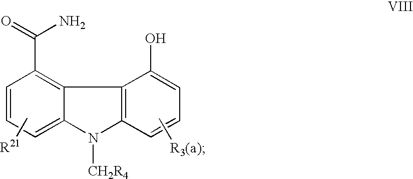
g) alkylating a compound of formula VIII with an alkylating agent of formula XCH₂R¹⁵, where X is halo and R¹⁵ is —CO₂R¹⁶, —SO R¹⁶, P(O) (OR¹⁶)₂, or —P(O)(OR¹⁶)H, where R¹⁶ is an acid protecting group to form a compound of formula VII 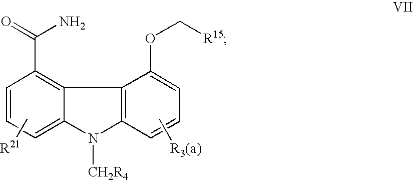
h) hydrolyzing a compound of formula VII to form a compound an acid or salt; and
i) converting the acid or salt of step (h) to form a compound of formula I.

23. A compound which is selected from the group consisting of;
[[5-Carbamoyl-9-(phenylmethyl)carbazol-4-yl]oxy]acetamide 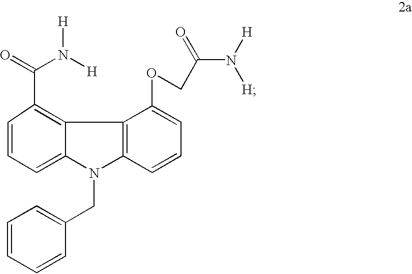
[[5-Carbamoyl-9-(phenylmethyl)carbazol-4-yl]oxy]-N-(ethyl)acetamide 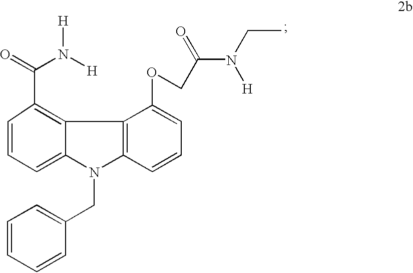
N-[[[5-Carbamoyl-9-(phenylmethyl)carbazol-4-yl]oxy]acetyl]glycine 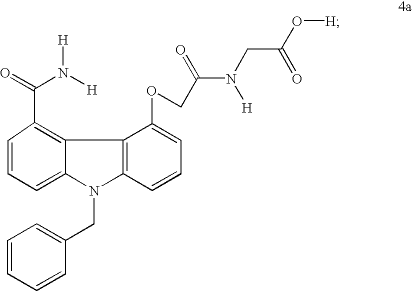
N-[[[5-Carbamoyl-9-(phenylmethyl)carbazol-4-yl]oxy]acetyl]glycine methyl ester 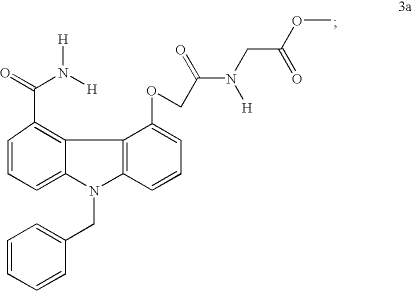
N-[[[5-Carbamoyl-9-(phenylmethyl)carbazol-4-yl]oxy]acetyl]glycine 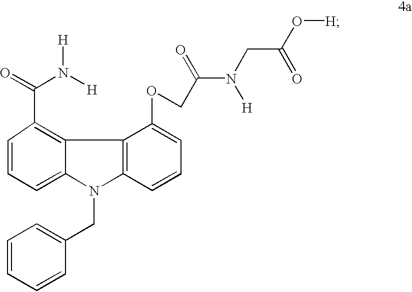
N-[[[5-Carbamoyl-9-(phenylmethyl)carbazol-4-yl]oxy]acetyl]-L-alanine 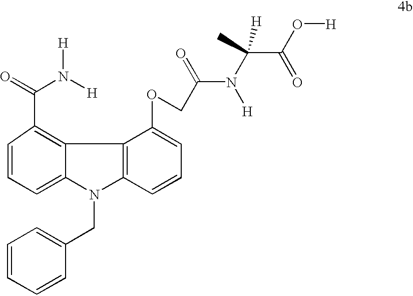
N-[[[5-Carbamoyl-9-(phenylmethyl)carbazol-4-yl]oxy]acetyl]-L-alanine methyl ester 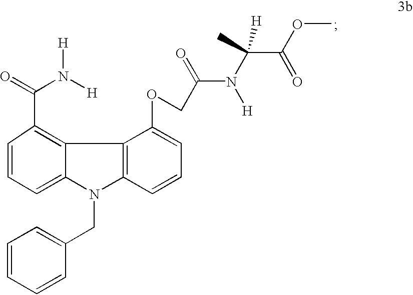
N-[[[5-Carbamoyl-9-(phenylmethyl)carbazol-4-yl]oxy]acetyl]-L-alanine 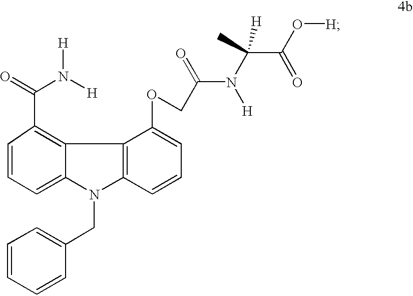
N-[[[5-Carbamoyl-9-(phenylmethyl)carbazol-4-yl]oxy]acetyl]-L-leucine 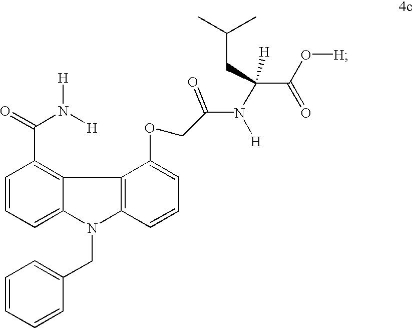
N-[[[5-Carbamoyl-9-(phenylmethyl)carbazol-4-yl]oxy]acetyl]-L-leucine methyl ester 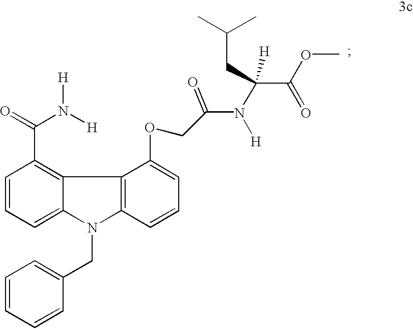
N-[[[5-Carbamoyl-9-(phenylmethyl)carbazol-4-yl]oxy]acetyl]-L-leucine 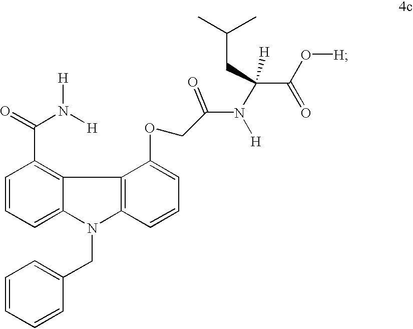
N-[[[5-Carbamoyl-9-(phenylmethyl)carbazol-4-yl]oxy]acetyl]-L-aspartic acid 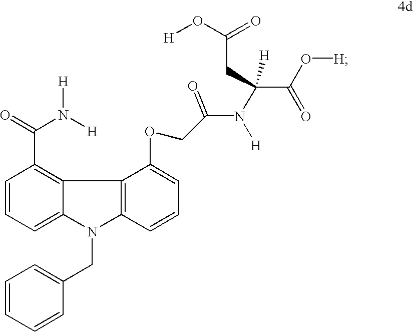
N-[[[5-Carbamoyl-9-(phenylmethyl)carbazol-4-yl]oxy]acetyl]-L-aspartic acid dimethyl ester 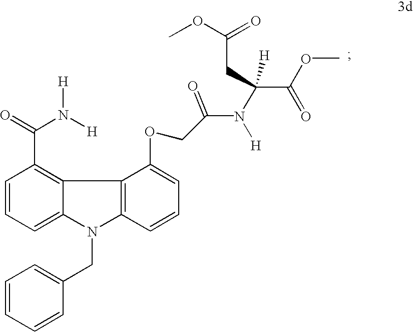
N-[[[5-Carbamoyl-9-(phenylmethyl)carbazol-4-yl]oxy]acetyl]-L-aspartic acid 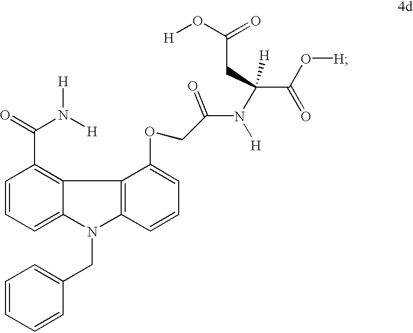
N-[[[5-Carbamoyl-9-(phenylmethyl)carbazol-4-yl] oxy] acetyl]-L-glutamic acid 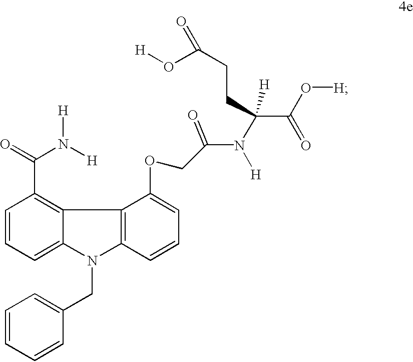
N-[[[5-Carbamoyl-9-(phenylmethyl)carbazol-4-yl]oxy]acetyl]-L-glutamic acid dimethyl ester 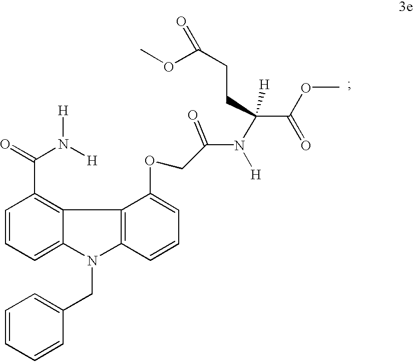
N-[[[5-Carbamoyl-9-(phenylmethyl)carbazol-4-yl]oxy]acetyl]-L-glutamic acid 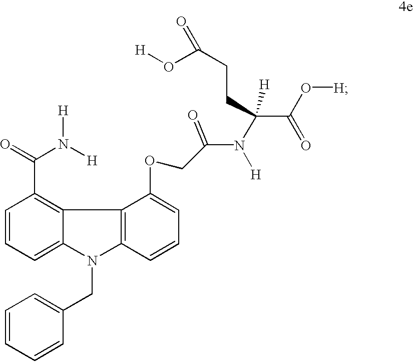
N-[[[5-Carbamoyl-9-(phenylmethyl)carbazol-4-yl]oxy]acetyl]-L-methionine 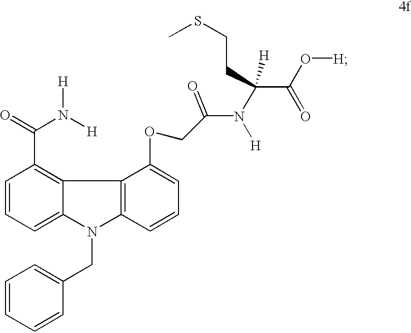
N-[[[5-Carbamoyl-9-(phenylmethyl)carbazol-4-yl]oxy]acetyl]-L-methionine methyl ester 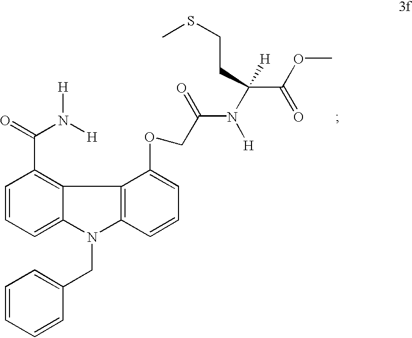
N-[[[5-Carbamoyl-9-(phenylmethyl) carbazol-4-yl]oxy]acetyl]-L-methionine 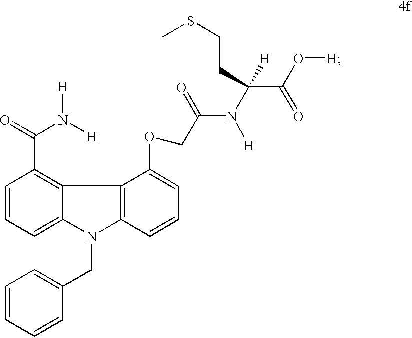
N-[[[5-Carbamoyl-9-(phenylmethyl)carbazol-4-yl]oxy]acetyl]-L-phenylalanine 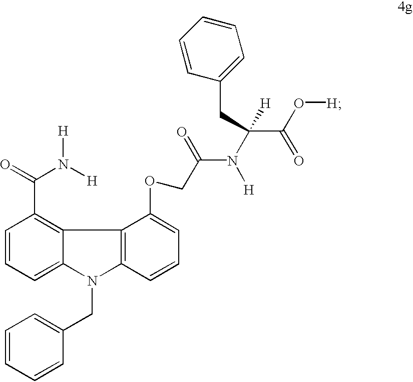
N-[[[5-Carbamoyl-9-(phenylmethyl)carbazol-4-yl]oxy]acetyl]-L-phenylalanine methyl ester 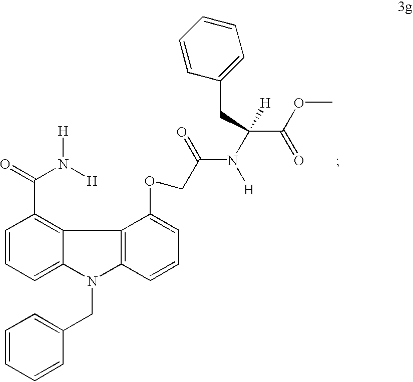
N-[[5-Carbamoyl-9- (phenylmethyl) carbazol-4-yl]oxy]acetyl]-L-phenylalanine 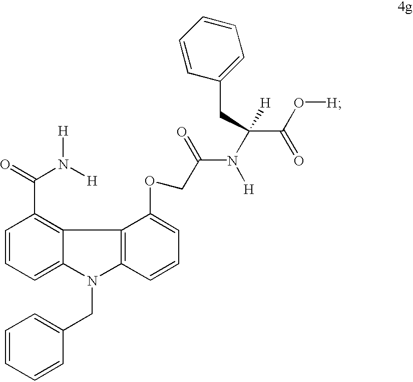
or a pharmaceuticallt acceptable racemate, solvate, tautomer, optical isomer, prodrug derivative or salt thereof.

24. The use of a compound of formula I as claimed in claim 1 for the manufacture of a medicament for the treatment of sepsis, septic shock, adult respiratory distress syndrome, pancreatitis, trauma-induced shock, bronchial asthma, allergic rhinitis, rheumatoid arthritis, cystic fibrosis, stroke, acute bronchitis, chronic bronchitis, acute bronchiolitis, chronic bronchiolitis, osteoarthritis, gout, spondylarthropathris, ankylosing spondylitis, Reiter's syndrome, psoriatic arthropathy, enterapathric spondylitis, Juvenile arthropathy or juvenile ankylosing spondylitis, Reactive arthropathy, infectious or post-infectious arthritis, gonoccocal arthritis, Tuberculous arthritis, viral arthritis, fungal arthritis, syphilitic arthritis, Lyme disease, arthritis associated with “vasculitic syndromes”, polyarteritis nodosa, hypersensitivity vasculitis, Luegenec's granulomatosis, polymyalgin rheumatica, joint cell arteritis, calcium crystal deposition arthropathris, pseudo gout, non-articular rheumatism, bursitis, tenosynomitis, epicondylitis (tennis elbow), carpal tunnel syndrome, repetitive use injury (typing), miscellaneous forms of arthritis, neuropathic joint disease (charco and joint), hemarthrosis (hemarthrosic), Henoch-Schonlein Purpura, hypertrophic osteoarthropathy, multicentric reticulohistiocytosis, arthritis associated with certain diseases, surcoilosis, hemochromatosis, sickle cell disease and other hemoglobinopathries, hyperlipoproteineimia, hypogammaglobulinemia, hyperparathyroidism, acromegaly, familial Mediterranean fever, Behat's Disease, systemic lupus erythrematosis, or relapsing polychondritis; and related diseases which comprises administering to a mammal in need of such treatment a therapeutically effective amount of a compound of formula I.
